SEQUENCE LISTING (8 sequences)

SEQ ID NO: 1 - DNA, 27 bp, Artificial Sequence
  Description of Artificial Sequence  Synthetic Primer
atctagatgggcaacttgaagagcgtg

SEQ ID NO: 2 - DNA, 26 bp, Artificial Sequence
  Description of Artificial Sequence  Synthetic Primer
aaccggggtggaaagcttgagggctc

SEQ ID NO: 3 - DNA, 63 bp, Artificial Sequence
  Description of Artificial Sequence  Synthetic Primer
agtaacatgggcaacttgaagagcgtgggcccctgagagccctctcgctttccaccccggttc

SEQ ID NO: 4 - DNA, 63 bp, Artificial Sequence
  Description of Artificial Sequence  Synthetic Primer
tcattgtacccgttgaacttctcgcacccggggactctcgggagagcgaaaggtggggccaag

SEQ ID NO: 5 - DNA, 66 bp, Artificial Sequence
  Description of Artificial Sequence  Synthetic Primer
tctagaatgcaccatcaccatcaccatggatatggacgtaaaaagcggaggcaacggcgcaggggg

SEQ ID NO: 6 - DNA, 66 bp, Artificial Sequence
  Description of Artificial Sequence  Synthetic Primer
agatcttacgtggtagtggtagtggtacctatacctgcatttttcgcctccgttgccgcgtccccc

SEQ ID NO: 7 - protein, 20 aa, Artificial Sequence
  Description of Artificial Sequence  Synthetic Peptide
MHHHHHHGYGRKKRRQRRRG

SEQ ID NO: 8 - protein, 11 aa, Artificial Sequence
  Description of Artificial Sequence  Synthetic Peptide
YGRKKRRQRRR

(19) United States
(12) Patent Application Publication
(10) Pub. No.: US 2005/0074448 A1
(43) Pub. Date: Apr. 7, 2005

(54) Method of treatment of endothelial dysfunction and engineered proteins for same

(75) Inventors: Elmer M. Price, Columbia, MO (US); Harold Laughlin, Columbia, MO (US); Chris Woodman, Columbia, MO (US); Miles Tanner, Columbia, MO (US); Michael Sturek, Zionsville, IN (US)

Correspondence Address: FULBRIGHT & JAWORSKI L.L.P., 600 CONGRESS AVE., SUITE 2400, AUSTIN, TX, 78701, US

(73) Assignee: The Curator sof the University of Missouri

(21) Appl. No.: 10/808,248

(22) Filed: Mar. 24, 2004

Related U.S. Application Data
(60) Provisional application No. 60/457,136, filed on Mar. 24, 2003.

(51) Int. Cl. A61K 38/44; G01N 33/53

(52) U.S. Cl. 424/94.4; 435/7.1

(57) ABSTRACT

Endothelial cells produce many factors that are involved in maintaining blood vessels. One of the best understood and most studied factor is nitric oxide (NO), which is produced by an endothelial enzyme called nitric oxide synthase (eNOS). The present invention provides a molecule designed to be delivered to the endothelium where it can reconstitute eNOS activity and restore NO production. Thus, the present invention provides methods of detecting, treating and preventing diseases or disorders associated with the vasculature and denoted by reduced levels of eNOS in the endothelium, as well as a concomitant reduction in the amount of NO that can be produced.

The present invention claims the benefit of the filing date of U.S. Provisional Patent Application Ser. No. 60/457,136 filed Mar. 24, 2003.

The government owns rights in the present invention pursuant to grant number HL52490 from the National Institutes of Health.

### BACKGROUND OF THE INVENTION

1. Field of the Invention

The present invention relates generally to the fields of protein therapy and vascular diseases. More particularly, it concerns the use of endothelial nitric oxide (eNOS) in the treatment and prevention of endothelial and vascular disorders.

2. Description of Related Art

Endothelial cells form a tight monolayer that lines the lumen of blood vessels. These cells produce a number of factors (e.g., nitric oxide (NO), endothelin, prostaglandins) which interact with the vascular smooth muscle cells of a blood vessel and serve to control, among other things, vasodilation and vasoconstriction. A functional endothelium is important in clotting, inflammation, and growth or cellular changes of the vascular smooth muscle cells (Escandon-Calles and Cipolla, 2001). Endothelial dysfunction has been implicated in a host of diseases and conditions such as hypertension (Olsen et al., 2001), atherosclerosis (Suwaidi et al., 2000; Hingorani, 2000; Luscher, 2001), diabetes (Escandon-Calles and Cipolla, 2001; De Vriese et al., 2000; Guerci et al., 2001a; Guerci et al., 2001b), hyperlipidemia (Ferrario et al., 2002; Maeso et al., 2000), and heart failure (Farre and Casado, 2001). In the Western world, hypertension and hypercholesterolemia are two major risk factors that can lead to atherosclerosis. Subsequently, atherosclerosis may result in a number of severe cardiovascular diseases, such as chronic heart failure, angina pectoris, claudicatio intermittens, or peripheral and myocardial ischemia.

One common characteristic of endothelial dysfunction as noted in related diseases or conditions, is the reduced levels of endothelial nitric oxide synthase (eNOS) in the endothelium and the subsequent reduction in the amount of nitric oxide (NO). Thus, therapies that increase the levels of eNOS and subsequent production of NO are needed in treating and preventing vascular or endothelial dysfunction.

Current therapies to enhance NO levels in the vasculature have been either to administer high doses of L-arginine (the eNOS substrate) or compounds such as nitroglycerine or sodium nitroprusside, which metabolically release NO. Although, these therapies can be effective, each has shown undesirable side-effects. Additionally, these therapies suffer from the inability to maintain a sustained release of NO, due to their rapid clearance from the body. Another drawback of nitroglycerin or sodium nitroprusside therapies is the inability of these agents to target specific vascular beds or regulate the release of NO.

Other approaches for increasing eNOS levels in the vasculature employ the use of exercise or gene therapy. Although NO levels have been shown to increase in trained/athletic individuals (Woodman et al., 1997), this approach has not been very effective since it requires maintenance of a vigorous exercise program. In addition, it is not clear that once the disease process has begun, the blood vessels will respond to exercise in the form of increases in endogenous eNOS expression. Furthermore, it is likely that exercise would not produce enough NO to reverse a endothelial or vascular disorder.

Gene therapy is also a current approach for treating vascular diseases with eNOS. These methods have been used to transfer eNOS cDNA to the endothelium (Claudius et al., 2002; Bivalacqua et al., 2000; Alexander et al., 2000; O'Brien et al., 1999; Qian et al., 2000; Cable et al., 1997a; Cable et al., 1997b; Lin et al., 1997). Viral or carrier-mediated delivery of eNOS cDNA into whole animals or tissues has been used to improve vascular tone in a variety of disease models. The delivery has been shown to enhance erectile function in the aged rat, suggesting functional expression of the eNOS cDNA (Bivalacqua et al., 2000). In terms of reversing disease-associated endothelial dysfunction, adenoviral eNOS constructs were effective at increasing NO bioavailability in spontaneously hypertensive rats (Alexander et al., 2000) and providing a prolonged reduction in high blood pressure in these animals (Lin et al., 1997). Gene therapy of eNOS also was shown to reduce adhesion molecule expression and cell infiltration in hypercholesterolemic rabbits (Qian et al., 2000).

However, these types of gene therapies have not proven efficacious in treating endothelial dysfunction. In general, gene therapy for treating vascular diseases or conditions have suffered from drawbacks such as biosafety, immunogenicity of viral vectors, optimization of vectors and promoters, low efficiency of liposome systems, duration of exogenous eNOS activity (it may not be advantageous or safe to have prolonged chronic recombinant NOS over-expressed in tissues), difficulty of large-scale production, and cellular delivery. Thus, new therapies to treat vascular diseases are needed.

### SUMMARY OF THE INVENTION

The present invention overcomes the deficiencies in the art for treating or preventing vascular diseases. A common characteristic of endothelial dysfunction and conditions or diseases associated with the vasculature is the reduced levels of endothelial nitric oxide synthase and the subsequent reduction in nitric oxide production. Thus novel, non-viral, non-DNA based therapies that increase the levels of eNOS and the subsequent production of NO in endothelial cells, would be beneficial as a therapeutic.

The present invention therefore provides a method for treating a vascular disease or condition comprising providing to a cell an endothelial nitric oxide synthase (eNOS) comprising a TAT protein transduction domain, wherein the eNOS increases nitric oxide production in a cell. In particular embodiments, it is contemplated that the endothelial nitric oxide synthase (eNOS) may comprise of the combination of a TAT protein transduction domain and a hexa-histidine domain. The vascular disease(s) or condition(s) to be treated may be a heart disease, hypertension, diabetes, atherosclerosis, hyperlipidemia, erectile dysfunction, arthritis, or any cardiovascular or endothelial disease or disorder in which the level of eNOS is reduced and subsequently the amount of NO. In particular embodiments of the invention, the cell is a vascular cell such as an endothelial cell or a vascular smooth muscle cell. In further embodiments, the cell may be located in a cell culture, a tissue culture or in the vasculature of a mammal.

In a further particular embodiment, the present invention provides a method for detecting a vascular disease in a subject comprising (a) obtaining a vascular cell sample from a subject; and (b) analyzing the cell sample for nitric oxide production by endothelial nitric oxide synthase, wherein a decrease in nitric oxide production as compared to a vascular cell sample from a healthy subject, indicates a vascular disease. In a further embodiment, the subject is a mammal such as a human. In yet another embodiment, the vascular cell sample is a vascular smooth muscle cell sample, a heart disease cell sample, a hypertension cell sample, a diabetes cell sample, a atherosclerosis cell sample or a hyperlipidemia cell sample.

In yet another embodiment, the present invention provides a method of analyzing a cell sample for nitric oxide production using a nitrite or a nitrate assay, a cGMP assay, or by monitoring blood pressure, blood flow, and improvements in vascular reactivity in a subject.

In a further embodiment, the present invention provides a method for detecting eNOS comprising a TAT protein transduction domain, or eNOS comprising a TAT protein transduction domain and a hexa-histidine domain, in the vascular cells, using immunoblotting. A hexa-histidine specific antibody may be used to detect the expression of eNOS protein.

In still yet another particular embodiment, the present invention provides a method for treating a subject having a vascular disease or condition comprising administering to the subject a therapeutic effective amount of an endothelial nitric oxide synthase comprising a TAT protein transduction domain. Vascular diseases or conditions contemplated include, but are not limited to, heart disease, hypertension, diabetes, atherosclerosis, hyperlipidemia, erectile dysfunction, arthritis, or any cardiovascular or endothelial disease or disorder.

The endothelial nitric oxide synthase comprising a TAT protein transduction domain, or endothelial nitric oxide synthase comprising a TAT protein transduction domain and a hexa-histidine domain may be administered intravenously, intraarterially, intramuscularly, intraperitoneally, subcutaneously, orally, as a suppository, or topically to a subject such as a mammal.

In still yet a further embodiment, the present invention provides a method for assessing the efficacy of eNOS comprising a TAT protein transduction domain as a vascular cell therapy comprising (a) administering an endothelial nitric oxide synthase protein comprising a TAT protein transduction domain to a subject having a vascular disease or condition; and (b) determining nitric oxide production; wherein an increase in the nitric oxide production as compared to the nitric oxide production in a vascular cell prior to administering eNOS comprising a TAT protein transduction domain, indicates that the vascular cell therapy is effective.

In yet another embodiment the present invention provides a method for determining nitric oxide production in a cell sample by a nitrite or a nitrate assay, or a cGMP assay, or by monitoring blood pressure, blood flow, and improvements in vascular reactivity in a subject.

In a further embodiment, the present invention provides a method for determining expression of eNOS comprising a TAT protein transduction domain, in vascular cells, using immunoblotting. In some embodiments the expression of eNOS comprising a TAT protein transduction domain may further comprise a hexa-histidine domain.

In still a further embodiment, the present invention provides an endothelial nitric oxide synthase comprising a TAT protein transduction domain or a pharmaceutical composition thereof. In further embodiments the endothelial nitric oxide synthase comprising a TAT protein transduction domain may further comprise a hexa-histidine domain.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. PCR™ cloning of porcine eNOS. The indicated forward and reverse primers were used to amplify cDNA made from aortic endothelium. The primers introduced unique restriction enzymes sites which were used for subcloning the amplicons into Bluescript.

FIG. 2. Engineering of His-TAT-tagged eNOS. Two complimentary oligos of the indicated sequences were annealed and ligated into the blunt-ended Xba I site of pBS-NOS. The cassette contains a new Xba I at the 5′ end, which destroys the original Xba I site and adds a new one such that a Xba I/Hind III double digest excises the intact HT-NOS cDNA.

FIG. 3. SDS-PAGE analysis of purified HT-NOS. The purified material is estimated to be about 85-90% pure based on densitometric (NIH Image) analysis of the digital image.

FIG. 4. Activity of purified HT-NOS. Two μg of purified material was assessed for activity via a radioactive arginine to citrulline assay. Duplicate data points were taken. The average calculated activity is shown.

FIG. 5. Nitrate/Nitrite analysis in media “conditioned” by cells transduced for 48 hours.

FIG. 6. Acetylcholine-mediated vasorelaxation of aortic rings from HT-NOS injected into rats. Sprague-Dawley rats were injected i.p. with 100 μg HT-NOS in 1 ml of 10% glycerol/PBS, or with 1 ml of carrier alone (control). Twenty-four hours after injection, aortic rings were isolated and analyzed for length-tension relationships in the presence of acetylcholine. The data shown is the average data of two rings per animal.

FIG. 7. Linear representation of an engineered TAT-hexahistidine-tagged version of eNOS (HT-NOS).

FIG. 8. Concentration-dependent transduction of cultured human endothelial cells using HT-NOS.

### DESCRIPTION OF ILLUSTRATIVE EMBODIMENTS

### A. The Present Invention

It is known in the art that NO is a key mediator of endothelial function (Busse and Flemin, 1999) and the inability of endothelial cells to produce adequate amounts of NO is a hallmark of endothelial dysfunction. Endothelial dysfunction leads to diseases or disorders such as, but not limited to, cardiovascular diseases, atherosclerosis, vasospasm, thrombosis restenosis, essential hypertension and diabetes. Clinically there is a need for NO therapy in preventing and treating endothelial dysfunction. A beneficial therapeutic approach for diseases or conditions associated with endothelial dysfunction involves increasing the amount of NO (via increasing NOS) produced by the endothelium. Therefore, the present invention provides eNOS as a therapeutic agent to increase NO production in vascular cells, thereby treating endothelial disorders.

The present invention therefore provides methods for detecting, preventing and treating vascular diseases in a subject. Central to the present invention is that deficiencies in eNOS lead to vascular dysfunction and that increasing the amount of eNOS protein and, hence, NO levels in the vasculature overcomes such deficiencies. The present invention also provides a molecule that can compensate for such reductions in endogenous eNOS levels and the subsequent attenuated NO production. The present invention provides a novel engineered version of recombinant eNOS that itself can be introduced into vascular tissue. This recombinant version of eNOS, designated “HT-NOS,” was engineered to contain a protein transduction domain, thereby facilitating its ability to be internalized by cells. In particular, the present invention comprises an eNOS protein tagged to an HIV TAT protein transduction domain comprising 11 amino acids of SEQ ID NO:8, which increases the production of NO in a vascular disease.

The eNOS of the present invention provides several advantages over the prior art. Recombinant NOS has been shown to correct certain aspects of endothelial dysfunction in animal studies. For example, there are no viral vectors involved in the present invention, hence biosafety and immunogenicity are not issues. Since TAT-tagged eNOS is itself being delivered, there is no component of gene expression involved, hence promoter choice is not an issue. TAT-tagged eNOS transduction can approach 100% efficiency, surpassing the predicted efficiencies of many gene therapy vectors. TAT-tagged eNOS has an finite life-span, therefore chronic long-term duration of treatment is not necessary since treatment for a specific time period would correct the endothelial or vascular disorder and thereby allow the endothelial or vascular cell to produce its own eNOS. However, if longer-term activity is necessary, TAT-tagged eNOS may be re-administered to achieve prolonged NO production. Recombinant TAT-tagged eNOS can be readily and routinely produced in large amounts, unlike viral production which is difficult to scale-up.

The present invention provides a vascular therapy, TAT-tagged eNOS, that may be used in conjunction with other therapies such as a second vascular therapy or a chemotherapy. The present invention further contemplates that some cancers, especially solid tumors, may respond better to chemotherapy when co-administered with TAT-tagged eNOS, which serves to enhance blood flow to the tumor mass, thereby improving the anti-cancer drug's bioavailability.

### B. Vascular Physiology of NO

Once synthesized in the endothelium, NO may freely diffuse to the underlying vascular smooth muscle or out into the vascular lumen, where it has the potential to interact with cellular elements such as thrombocytes and leukocytes. Alternatively, NO may act in an autocrine or paracrine manner within the endothelium. On the other hand, NO may simply be sequestered by binding to albumin or erythrocyte-associated hemoglobin in the vascular lumen; or may face inactivation following oxidation or reaction with reactive oxygen species such as superoxide anion. The nondegradative interactions of NO represent physiological signaling events which, depending on the target cell, mediate vasodilation, antivasoconstriction and antiproliferation (vascular smooth muscle); reduced permeability (endothelium); antiaggregation (thrombocytes); and antiadhesion (leukocytes). These intracellular actions of NO predominantly arise from the activation of soluble guanylyl cyclase (the intracellular receptor for NO) and subsequent generation of the second messenger cGMP, intracellular levels of which are strictly regulated by phosphodiesterase type V (PDE V) activity.

### C. Endothelail Nitric Oxide Synthase

As discussed above, nitric oxide (NO) is an important pleiotropic mediator of many biological responses. NO is derived from the amino acid L-arginine. Nitric oxide synthase (NOS) catalyzes NADPH-dependent conversion of arginine to citrulline and NO. NOS acts upon arginine to oxidize one of the guanidino nitrogens to NO.

Three isoforms of NOS, which roughly define three areas of NO involvement, have been described. They are endothelial cell (eNOS, blood pressure regulation), brain or neuronal (bNOS, neurotransmitter activity), and macrophage or inducible (iNOS, cytostatic and cytotoxic activity against pathogens). eNOS shares about 50% amino acid sequence homology with the two other NOS isozymes. eNOS, as the other isozymes, appears to be comprised of homodimers with subunit of molecular weights of 125-150 kD. NOS isozymes appear to make NO by the same overall mechanism (Marietta, 1993.) The enzyme mechanism most likely involves passage of electrons from NADPH through FMN/FAD to heme and then to arginine. Calmodulin binding seems to act as a trigger to allow electrons to flow from the flavins to the heme. The carboxy-terminal half shows homology to cytochrome P450 reductase with binding sites identified by homology for FMN, FAD, and NADPH. The amino terminal region is not homologous to any other protein and probably contains binding sites for heme, arginine and tetrahydrobiopterin (THB). A calmodulin binding site is found near the middle of all three NOS enzymes.

Thus, NOS isozymes are generally classified into two distinct categories or systems: a constitutively expressed calcium/calmodulin-dependent system; and a cytokine-inducible calmodulin-independent system. These two systems exhibit differences in regulation of expression, cofactor dependence, tissue distribution and subcellular localization.

Endothelial NOS is a constitutive enzyme of limited tissue distribution which is present in low amounts. The activity of eNOS is regulated by calcium/calmodulin. Constitutive production of nanomolar amounts of NO by endothelial cells appears to be vital to the regulation of homeostasis. Additionally, constitutive production of NO is critical for signal transduction in the central nervous system. Activation of guanylyl cyclase by NO is one of the major targets of the constitutive NOS system. Activation of guanylate cyclase increases the level of cGMP and causes the smooth cell to relax, thus dilating the vessel and increasing the blood flow (Moncada et al., 1993; Vallance, et al., 1994).

### D. Protein Transduction Domain

Protein transduction is a process by which peptides or proteins cross the cellular plasma membrane. One of the main advantages of protein transduction is its applicability in a variety of cells, even cells that are difficult to transfect with DNA. In addition, the speed of delivery (the protein is detected in minutes rather than several hours or days as with DNA); the ease of translocation across the plasma membrane; and the lack of toxicity make protein transduction a useful technique.

The three most widely studied protein transduction domains are derived from the Drosophila homeotic transcription factor Antennapedia (Antp), the herpes simplex virus (HSV) protein VP22, and the human immunodeficiency virus (HIV)-1 transcriptional activator TAT. Other protein transduction domains include the Drosophila homeotic factors Fushi-tarazu and Engrailed, which contain peptides with similar properties as Antp, VP22 and TAT (Han et al., 2000).

Protein transduction domains are generally short peptides, about 10-16 residues in length. These domains, although structurally dissimilar, have a common characteristic of numerous positively charged lysine and arginine residues (Schwarze et al., 2000). Protein transduction mediated by these peptides appears to-be independent of the protein size. For example, these peptides have been shown to deliver covalently attached proteins in excess of 700 kDa to cells. They have also transduced liposomes over 200 nm in diameter, about the size of a mitochondrion, directly across the cell membrane by anchoring protein transduction domains to the liposome surface (Torchilin et al., 2001).

The present invention therefore contemplates the use of the HIV TAT protein transduction domain tagged to eNOS in order to facilitate the ability of eNOS internalization into the cells. In particular, the present invention contemplates the use of the TAT protein transduction domain comprising 11 amino acids of SEQ ID NO:8. Purified human immunodeficiency virus type-1 (HIV) TAT protein has been shown to be taken up from the surrounding medium by human cells growing in culture (Frankel and Pabo, 1988). The TAT protein trans-activates certain HIV genes and is essential for viral replication. The full-length HIV-1 TAT protein contains 101 amino acid residues. The HIV TAT gene has two exons. Amino acids 1-72 are encoded by exon 1, and amino acids 73-86 are encoded by exon 2. The full-length TAT protein is characterized by a basic region which contains an arginine rich stretch of nine amino acids embedded within the protein. This basic region of the TAT protein is thought to be important for nuclear localization (Ruben et al., 1989; Hauber et al., 1989). The cysteine-rich region contains seven cysteine residues (amino acids 22-37) and mediates the formation of metal-linked dimers in vitro (Frankel et al., 1988a; Frankel et al., 1988b) and is essential for its activity as a transactivator (Garcia et al., 1988; Sadaie et al., 1989). As in other regulatory proteins, the N-terminal region of the TAT protein may be involved in protection against intracellular proteases (Bachmair et al., 1989).

### E. eNOS Comprising a Protein Transduction Domain

The present invention contemplates endothelial nitric oxidase synthase (eNOS) comprising a protein transduction domain for treating vascular diseases or conditions. Thus, the present invention concerns a novel composition of eNOS comprising a protein transduction domain engineered into its amino terminus. The terms “protein,” “polypeptide” and “peptide” may be used interchangeably in describing eNOS and the protein transduction domain sequences of the present invention.

As used herein, an “amino acid molecule” or “amino acid residue” refers to any naturally occurring amino acid, any amino acid derivative or any amino acid mimic known in the art, including modified or unusual amino acids. In certain embodiments, the residues of the protein or peptide are sequential, without any non-amino acid interrupting the sequence of amino acid residues. In other embodiments, the sequence may comprise one or more non-amino acid moieties. In particular embodiments, the sequence of residues of the protein or peptide may be interrupted by one or more non-amino acid moieties. In specific aspects, the composition of the present invention employs a protein of greater than about 200 amino acids or the full length endogenous sequence translated from a gene; a polypeptide of greater than about 100 amino acids; and/or a peptide of from about 10 to about 100 amino acids.

Proteins or peptides may be made by any technique known to those of skill in the art, including the expression of proteins, polypeptides or peptides through standard molecular biological techniques, the isolation of proteins or peptides from natural sources, or the chemical synthesis of proteins or peptides. The nucleotide and protein, polypeptide and peptide sequences corresponding to various genes have been previously disclosed, and may be found at computerized databases known to those of ordinary skill in the art. One such database is the National Center for Biotechnology Information's Genbank and GenPept databases located at the National Institutes of Health website on the Internet. The coding regions for known genes may be amplified and/or expressed using the techniques disclosed herein or as would be know to those of ordinary skill in the art. Alternatively, various commercial preparations of proteins, polypeptides and peptides are known to those of skill in the art.

In particular aspects, the present invention concerns a composition comprising a eNOS protein the size of which may comprise, but is not limited to, about 10, about 15, about 20, about 25, about 30, about 31, about 32, about 35, about 40, about 45, about 50, about 55, about 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, about 250, about 300, about 350, about 400, about 450, about 500, about 550, about 600, about 650, about 700, about 750, about 800, about 850, about 900, about 950, about 1000, about 1100, about 1200, about 1300, about 1400, about 1500, about 1750, about 2000, about 2250, about 2500 or greater amino molecule residues, and any range derivable therein.

The composition of the present invention encompasses amino molecule sequences comprising at least one of the 20 common amino acids in naturally synthesized proteins, or at least one modified or unusual amino acid, including but not limited to those shown in Table 1 below.

TABLE 1
Modified and Unusual Amino Acids

| Abbr. | Amino Acid |
|---|---|
| Aad | 2-Aminoadipic acid |
| Baad | 3-Aminoadipic acid |
| Bala | -alanine, -Amino-propionic acid |
| Abu | 2-Aminobutyric acid |
| 4Abu | 4-Aminobutyric acid, piperidinic acid |
| Acp | 6-Aminocaproic acid |
| Ahe | 2-Aminoheptanoic acid |
| Aib | 2-Aminoisobutyric acid |
| Baib | 3-Aminoisobutyric acid |
| Apm | 2-Aminopimelic acid |
| Dbu | 2,4-Diaminobutyric acid |
| Des | Desmosine |
| Dpm | 2,2′-Diaminopimelic acid |
| Dpr | 2,3-Diaminopropionic acid |
| EtGly | N-Ethylglycine |
| EtAsn | N-Ethylasparagine |
| Hyl | Hydroxylysine |
| Ahyl | allo-Hydroxylysine |
| 3Hyp | 3-Hydroxyproline |
| 4Hyp | 4-Hydroxyproline |
| Ide | Isodesmosine |
| Aile | allo-Isoleucine |
| MeGly | N-Methylglycine, sarcosine |
| MeIle | N-Methylisoleucine |
| MeLys | 6-N-Methyllysine |
| MeVal | N-Methylvaline |
| Nva | Norvaline |
| Nle | Norleucine |
| Orn | Ornithine |

In further embodiments the eNOS composition of the present invention comprises a biocompatible protein, polypeptide or peptide. As used herein, the term “biocompatible” refers to a substance which produces no significant untoward effects when applied to, or administered to, a given organism according to the methods and amounts described herein. Such untoward or undesirable effects are those such as significant toxicity or adverse immunological reactions. In preferred embodiments, biocompatible protein, polypeptide or peptide containing compositions will generally be mammalian proteins or peptides or synthetic proteins or peptides each essentially free from toxins, pathogens and harmful immunogens.

1. Generation of eNOS (HT-NOS) Fusion Protein

Other aspects of the present invention concern fusion proteins or peptides, which comprise the eNOS protein fused to a TAT and/or a hexa-histidine peptide, polypeptide, or protein. Such a fusion protein of the present invention may comprise all or a substantial portion of the eNOS protein, linked at the amino terminus, to all or a portion of a protein transduction domain or an additional peptide, polypeptide, or protein.

In the present invention, the delivery, or transduction, of a protein, such as NOS, into cells is mediated by the presence of a protein transduction domain engineered into the protein's amino-terminus. Such protein transduction domains are short specific peptides that can cross cell membranes and enter the cell (Nagahara et al., 1998). Furthermore, when coupled to larger proteins, these protein transduction domain peptide sequences will allow these larger macromolecules to similarly enter the cell. In particular embodiments of the invention, the TAT protein transduction domain comprising of 11 amino acids (SEQ ID NO:8) is fused to the amino-terminus of the eNOS protein.

Data in the literature reveals that a wide variety of proteins can be TAT-tagged and transduced into cells (Nagahara et al, 1998; Schwarze et al., 1999; Jin et al., 2001; Kwon et al., 2000; Darbinian et al., 2001; Yang et al., 2002). More than 50 different TAT-tagged proteins have been described as being transduced into cells, including proteins greater than 100 Kd in size. Transduced proteins are active once transduced into cells, and efficiencies approaching 100% are common (Nagahara et al., 1998). Schwarze et al. (1999) reported TAT-tagged β-galactosidase (a 120 Kd protein) transduction in cells in an intact, living mouse.

As used herein, a protein transduction domain such as a TAT domain of the present invention, refers to a relatively short specific peptide sequence. Typically, these peptides will be between about 10 amino acids to about 50 amino acids in length, however any peptide of any length that is useful in transporting proteins, such as eNOS, across cell membranes and into the cell, as described herein, is within the scope of this definition. Specifically, such peptides may be 5, 6, 7, 8, 9, 10, 11, or more amino acid residues in length. It is specifically contemplated that the invention relates to protein transduction domains that are of any length between 1 and 100 amino acid residues. Further, the inventive peptides may be described in terms of any range of lengths derivable between the integers disclosed above, for example but not limited to lengths between 5 and 10, 5 and 15, 10 and 30, and 20 and 40 amino acids.

Other examples of fusion proteins involves the use of linkers which may comprise bifunctional cross-linking reagents. Such linkers are known to those of skill in the art. In addition, fusion proteins may comprise leader sequences from other species to permit the recombinant expression of a protein in a heterologous host. Another example of fusion proteins includes the addition of an immunologically active domain, such as an antibody epitope, to facilitate purification of the fusion protein. Inclusion of a cleavage site at or near the fusion junction facilitates removal of the extraneous polypeptide after purification. A fusion protein of the present invention may also comprise a glycine amino acid fused between the TAT protein transduction domain and the eNOS protein, or between the hexa-histidine domain and the TAT domain, or between the hexa-histidine domain and the TAT domain and the eNOS protein. Such an amino acid provides added degrees of freedom required for activity.

Other useful fusions include linking of functional domains, such as active sites from enzymes, glycosylation domains, cellular targeting signals or transmembrane regions. Methods of generating fusion proteins are well known to those of skill in the art. For example, fusion proteins may be made by de novo synthesis of the complete fusion protein or by attachment of a nucleic acid sequence encoding the eNOS protein to a nucleic acid sequence encoding the second peptide or protein such as a protein transduction domain, followed by expression of the intact fusion protein.

2. Variants of the eNOS Protein

It is contemplated by the inventors that the peptides of the present invention may further employ amino acid sequence variants such as substitutional, insertional or deletion variants. Deletion variants lack one or more residues of the native protein. Insertional mutants typically involve the addition of material at a non-terminal point in the polypeptide. Substitutions are changes to an existing amino acid. These sequence variants may generate truncations, point mutations, and frameshift mutations. As is known to one skilled in the art, synthetic peptides can be generated by these mutations.

It also will be understood that amino acids sequence variants may include additional residues, such as additional N- or C-terminal amino acids, and yet still be essentially as set forth in one of the sequences disclosed herein, so long as the sequence meets the criteria set forth above, including the maintenance of biological activity.

The following is a discussion based upon changing the amino acids of a protein, such as a eNOS protein, to create a mutated, truncated, or modified protein. For example, certain amino acids may be substituted for other amino acids in the eNOS protein, resulting in a greater cell uptake, increased levels of eNOS and increase production of NO. Since it is the interactive capacity and nature of a protein that defines that protein's biological functional activity, certain amino acid substitutions can be made in a protein sequence, and in its underlying nucleic acid coding sequence, thereby producing a mutated, truncated or modified protein.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: basic amino acids: arginine (+3.0), lysine (+3.0), and histidine (−0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5±1), alanine (−0.5), and glycine (0); hydrophobic, aromatic amino acids: tryptophan (−3.4), phenylalanine (−2.5), and tyrosine (−2.3).

It is understood that an amino acid can be substituted for another having a similar hydrophilicity and produce a biologically or immunologically modified protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

The present invention may also employ the use of peptide mimetics for the preparation of polypeptides (see e.g., Johnson, 1993) having many of the natural properties of the eNOS protein, but with altered and/or improved characteristics. The underlying rationale behind the use of peptide mimetics is that the peptide backbone of proteins exists chiefly to orient amino acid side chains in such a way as to facilitate molecular interactions, such as those of antibody and antigen. These principles may be used, in conjunction with the principles outline above, to engineer second generation molecules having many of the natural properties of eNOS, but with altered and even improved characteristics.

3. Protein Purification

In certain embodiments the TAT and/or hexa-histidine tagged eNOS protein of the present invention may be purified. It may be desirable to purify eNOS, or variants thereof. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, antibodies and the like or by heat denaturation, followed by centrifugation; gel filtration, reverse phase, hydroxylapatite and affinity chromatography; and combinations of such and other techniques.

In purifying the TAT- and His-tagged eNOS of the present invention, it may be desirable to express the polypeptide in a prokaryotic or eukaryotic expression system and extract the protein using denaturing conditions. The polypeptide may be purified from other cellular components using a Ni-affinity column, which binds to the hexa-histidine tag of the polypeptide. This procedure results in a highly purified preparation of TAT- and His-tagged polypeptide. Although this preparation will be purified in an inactive form, the denatured material will still be capable of transducing cells. Once inside of the target cell or tissue, it is generally accepted that the polypeptide will regain full biological activity.

As is generally known in the art, it is believed that the order of conducting the various purification steps may be changed, or that certain steps may be omitted, and still result in a suitable method for the preparation of a substantially purified protein or peptide.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. Another method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

### F. Nucleic Acids Encoding eNOS Protein and Transduction Domain

It is contemplated in the present invention, that the eNOS protein comprising a protein transduction domain may be encoded by a nucleic acid sequence. A nucleic acid may be derived from genomic DNA, complementary DNA (cDNA) or synthetic DNA. Where incorporation into an expression vector is desired, the nucleic acid may also comprise a natural intron or an intron derived from another gene.

As used herein, the term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression or where non-coding regions such as introns are to be targeted in an antisense strategy. eNOS cDNA, for use in the present invention, may be derived from porcine or human cDNA but are not limited such.

As used herein, the term “nucleic acid segment” refers to a nucleic acid molecule that has been isolated free of total genomic DNA of a particular species. Therefore, a nucleic acid segment encoding a polypeptide refers to a nucleic acid segment that contains wild-type, polymorphic, or mutant polypeptide-coding sequences yet is isolated away from, or purified free from, total mammalian or human genomic DNA. Included within the term “nucleic acid segment” are a polypeptide or polypeptides, DNA segments smaller than a polypeptide, and recombinant vectors, such as, plasmids and other non-viral vectors.

The term “recombinant” may be used in conjunction with a polypeptide or the name of a specific polypeptide, and generally refers to a polypeptide produced from a nucleic acid molecule that has been manipulated in vitro or that is the replicated product of such a molecule. Recombinant vectors and isolated nucleic acid segments may variously include the eNOS-coding regions themselves, coding regions bearing selected alterations or modifications in the basic coding region, or they may encode larger polypeptides that nevertheless include eNOS-coding regions or may encode biologically functional equivalent proteins or peptides that have variant amino acids sequences.

A “nucleic acid” as used herein includes single-stranded and double-stranded molecules, as well as DNA, RNA, chemically modified nucleic acids and nucleic acid analogs. It is contemplated that a nucleic acid within the scope of the present invention may be of about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or greater nucleotide residues in length. Those of skill will recognize that in cases where the nucleic acid region encodes eNOS comprising a protein transduction domain, the nucleic acid region can be quite long, depending upon the number of amino acids in the fusion protein.

It is contemplated that the eNOS protein comprising a protein transduction domain may be encoded by any nucleic acid sequence that encodes the appropriate amino acid sequence. The design and production of nucleic acids encoding a desired amino acid sequence is well known to those of skill in the art, using standardized codon tables (Table 2). In preferred embodiments, the codons selected for encoding each amino acid may be modified to optimize expression of the nucleic acid in the host cell of interest. The term “functionally equivalent codon” is used herein to refer to codons that encode the same amino acid, such as the six codons for arginine or serine, and also refers to codons that encode biologically equivalent amino acids. Codon preferences for various species of host cell are well known in the art. Codons preferred for use in humans, are well known to those of skill in the art (Wada et.al., 1990). Codon preferences for other organisms also are well known to those of skill in the art (Wada et al., 1990, included herein in its entirety by reference).

TABLE 2
Codon Table

| Amino Acids | | | Codons |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

Prokaryote- and/or eukaryote-based systems can be used to produce nucleic acid sequences, or their cognate polypeptides, proteins and peptides. The present invention contemplates the use of such an expression system to produce the eNOS protein. More specifically, the present invention employs the use of the insect cell/baculovirus system. The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

In addition to the expression system disclosed in the invention, numerous expression systems exists which are commercially and widely available. One example of such a system is the STRATAGENE®'s COMPLETE CONTROL Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the flill-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

### G. Methods of Protein Delivery

The present invention also employs methods for delivering eNOS comprising a protein transduction domain such as TAT and/or hexa-histidine domain, or variants thereof to a cell, tissue or organism to increase the levels of eNOS and subsequently NO production. Such methods of delivery are believed to include virtually any method by which a protein can be introduced into an organelle, a cell, a tissue or an organism, as would be known to one of ordinary skill in the art. Methods contemplated in the present invention include, but are not limited to, direct delivery of the tagged eNOS to the vicinity of the cell, or via a catheter for delivery to a subject. For cultured cells, sterile solutions of eNOS comprising a TAT protein and/or a hexa-histidine tagged domain can be added to the medium in which the cells are grown. Under these circumstances, cells can internalize the TAT-tagged eNOS protein within about one minute of incubation. For delivery to a subject, sterile solutions of the TAT-tagged eNOS protein (as described herein) can be administered via a catheter directly into the blood stream, either intravenous, or intra-arterial, but is not limited to such. For subjects undergoing balloon angioplasty, or other similar procedure, TAT-tagged eNOS protein can be delivered directly to the affected artery via the balloon catheter.

### H. Thearapeutic Applications of eNOS

The present invention provides a therapeutic agent such as eNOS for the treatment and prevention of vascular diseases or conditions. The present invention has application of eNOS in a variety of diseases such as: heart disease, hypertension, diabetes, atherosclerosis, hyperlipidemia, angioplasty, aging, arthritis, erectile dysfunction and as an adjuvant for chemotherapy.

The treatment, prevention, and diagnosis of vascular conditions and diseases characterized by endothelial dysfunction are contemplated as part of the present invention. Endothelial dysfunction include disorders such as hyperlipidemia, arteriosclerosis, thrombosis, and restenosis and a variety of cardiovascular disorders but are not limited to such. A “cardiovascular disorder” refers to a disorder, disease, or condition which affects the cardiovascular system, e.g., the heart, the blood vessels, or the blood. Cardiovascular disorders can be characterized by an inadequate supply of blood to an organ, e.g., the heart; the accumulation of fatty substances, e.g., cholesterol or triglycerides, in the walls of blood vessels; an irregularity in the heart rhythm; or a defective conduction of impulses from the atria to the ventricles of the heart. For example, cardiovascular disorders include heart failure (chronic heart failure), hypertension, atherosclerosis, coronary artery disease, coronary artery spasm, arrythmias, atrial fibrillation, dilated cardiomyopathy, idiopathic cardiomyopathy, angina peripheral and myocardial ischemia. Diseases or disorders particularly contemplated for use with the compositions and methods of the present invention are atherosclerosis, diabetes, hyperlipidermia, angioplasty, heart disease, hypertension, restenosis, hemangioma, and cancer angiogenesis.

Atherosclerosis involves the deposit of fatty substances, cholesterol, cellular waste products, calcium and other substances in the inner lining of an artery. This accumulation is referred to as “plaque.” Arteriosclerosis, which often accompanies atherosclerosis, refers to the hardening of arteries. Plaque can partially or completely block blood flow through an artery, which may lead to a heart attack or stroke. Restenosis is an accelerated form of atherosclerosis in which hyperproliferation of smooth muscle cells in the vascular wall may quickly obstruct the lumen, which may have been enlarged by percutaneous transluminal coronary angioplasty (PTCA), stent placement, or atherectomy. The reduction in the bioavailability of NO caused by genetic or environmental (risk factors such as smoking, age, or hyperlipidemia, for example) greatly hastens the progression of atherosclerosis. It is known that NO has a protective role in fighting this disease by inhibiting cell growth, platelet aggregation, and cell adhesion, among other things. Thus, it is contemplated in the present invention that delivery of eNOS (HT-NOS) to the blood vessels would prevent or lessen the progression of atherosclerosis and related conditions.

Angioplasty is a procedure, used to remove or widen an artery blocked because of atherosclerosis, this usually leaves behind a damaged vessels that have been denuded of endothelial cells. The endothelial lining eventually grows back, but during this period of re-endothelialization the blood vessel is prone to spasms (spontaneous vasoconstrictions), which can be serious and life-threatening. Furthermore, inappropriate cell migration or growth can lead to a narrowing of the artery in the region of the angioplasty (thereby reversing the beneficial effect of the procedure). eNOS, delivered to the vascular smooth muscle cells of the denuded blood vessels during or just after angioplasty would reconstitute NO production and prevent these spasms as well as control restenosis. A recombinant eNOS such as provided in the present invention, would be used that has an increased basal activity relative to wildtype eNOS. This recombinant would contain the TAT tag sequence as described, plus contain an additional amino acid modification which is a serine to aspartate change at position 1177 in human eNOS (1179 in porcine eNOS). This will generate a form of eNOS that would be active in vascular smooth muscle cells.

Another application for eNOS therapy is diabetes. Diabetes is a disease manifested by several cellular abnormalities, one of which is endothelial dysfunction and its associated reduction in NO production. This defect in the vasculature results in atherosclerosis and subsequently patient morbidity and mortality. Thus, it is contemplated that NO produced by HT-NOS would reverse or attenuate the onset of atherosclerosis and hence, an improvement in the prognosis for individuals with diabetes.

HT-NOS of the present invention may also be used as a therapeutic agent in treating heart disease. Heart disease is considered the end result of a series of insults to the cardiac myocytes, which eventually leads to the death of these cells. The accumulation or activity of reactive oxygen species is thought to participate in this cell toxicity. NO, produced by the delivery of HT-NOS to these cells, would serve to scavenge the toxic molecules. Thus, patients diagnosed with heart failure, especially if diagnosed early, can be given HT-NOS to prevent further deterioration of the heart muscle.

The eNOS therapeutic agent of the present invention also has applicability in treating high blood pressure which is caused, in part, by an impaired ability of the blood vessels to vasodilate, and by changes in vascular structure. Reductions in NO production appear to be involved in both this loss in vasodilation (relaxation) as well as in the promotion of vascular remodeling. NO, produced by the action of administering eNOS, would protect against these changes that lead to hypertension.

Although the mechanism of action is not entirely clear, hyperlipidemia appears to reduce the bioavailability of NO in vascular tissues, possibly via the action of oxidized lipids. This lowered NO production leads to endothelial dysfunction and increases the propensity for the vessel to undergo atherogenesis. Thus, increasing the local availability of NO via the administration of eNOS would reduce or prevent the progression of this disease.

In other aspects, the therapeutic agent of the present invention may be used to treat tumors such as solid tumors which are often resistant to chemotherapy and therefore require surgery to remove the growth. One mechanism attributing to chemotherapeutic resistance in solid tumors is poor blood flow to the center portion of the tumor mass. Thus, it is contemplated in the present invention that a short term treatment with HT-NOS, delivered locally to the tumor, would increase blood flow to the mass, thereby increasing the anti-cancer efficacy of an administered chemotherapeutic agent.

Other aspects of the present invention provides HT-NOS as a therapeutic agent for vascular diseases associated with aging. Endothelial dysfunction appears to worsen with age, therefore HT-NOS is contemplated for use in slowing or preventing age-related vascular disorders.

### I. Formulations and Routes for Administration of eNOS

Where clinical applications are contemplated, it will be necessary to prepare pharmaceutical compositions in a form appropriate for the intended application. The preparation of a pharmaceutical composition that contains eNOS or additional active ingredient will be known to those of skill in the art in light of the present disclosure, and as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

Aqueous compositions of the present invention in an effective amount may be dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the present invention, its use in therapeutic compositions is contemplated. Supplementary active ingredients also can be incorporated into the compositions.

The composition(s) of the present invention may be delivered by a number of methods known to one of skill in the art. Preferably, the composition of the present invention may be delivered intravenously, intraarterially, intratumorally, intraperitoneally, subcutaneously, orally or topically. In some embodiments, local or regional delivery of eNOS, to a patient with a vascular condition will be a very efficient method for delivering a therapeutically effective gene to counteract the clinical disease.

Intra-arterial administration is achieved using a catheter that is inserted into an artery to an organ or to an extremity. Typically, a pump is attached to the catheter. Intracavity administration is applicable when therapeutic drugs are introduced directly into a body cavity such as intravesical (into the bladder), peritoneal (abdominal) cavity, or pleural (chest) cavity. Agents can be given directly via catheter. Intrapleural administration is accomplished using large and small chest catheters, while a Tenkhoff catheter (a catheter specially designed for removing or adding large amounts of fluid from or into the peritoneum) or a catheter with an implanted port is used for intraperitoneal delivery.

Alternatively, systemic delivery of the eNOS therapeutic agent may be appropriate in certain circumstances, for example, where the disease state or condition is extensive. Intravenous therapy can be implemented in a number of ways, such as by peripheral access or through a vascular access device (VAD). A VAD is a device that includes a catheter, which is placed into a large vein in the arm, chest, or neck. It can be used to administer several drugs simultaneously, for long-term treatment, for continuous infusion, and for drugs that are vesicants, which may produce serious injury to skin or muscle. Various types of vascular access devices are available.

The active compositions of the present invention may include classic pharmaceutical preparations. Administration of these compositions according to the present invention will be via any common route so long as the target tissue is available via that route. Alternatively, administration may be by intradermal, subcutaneous, intraperitoneal, intravenous injection, intratumoral, oral or local administration. Such compositions would normally be administered as pharmaceutically acceptable compositions, described supra. The drugs and agents also may be administered parenterally or intraperitoneally. The term “parenteral” is generally used to refer to drugs given intravenously, or subcutaneously.

Solutions of the active compounds as free base or pharmacologically acceptable salts can be prepared in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The therapeutic compositions of the present invention may be administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like. Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH, exact concentration of the various components, and the pharmaceutical composition are adjusted according to well known parameters. Suitable excipients for formulation with eNOS include croscarmellose sodium, hydroxypropyl methylcellulose, iron oxides synthetic), magnesium stearate, microcrystalline cellulose, polyethylene glycol 400, polysorbate 80, povidone, silicon dioxide, titanium dioxide, and water (purified).

In certain aspects of the invention, the eNOS composition may be prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain preferred embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings, e.g., enteric coatings or liposomes, or to otherwise modify the physical form of the dosage unit. For instance, tablets, pills, or capsules may be coated with shellac, sugar or both.

Enteric coatings may be applied to tablets to prevent the release of drugs in the stomach either to reduce the risk of unpleasant side effects or to maintain the stability of the drug which might otherwise be subject to degradation of expose to the gastric environment. Most polymers that are used for this purpose are polyacids that function by virtue of the fact that their solubility in aqueous medium is pH-dependent, and they require conditions with a pH higher then normally encountered in the stomach. Enteric coatings may be used to coat a solid or liquid dosage form of TAT-tagged eNOS. Enteric coatings allows the drug to remain physically incorporated in the dosage form for a specified period when exposed to gastric juices. Enteric coatings are designed to disintegrate in intestinal fluid for ready absorption. Typical enteric coating agents include, but are not limited to, hydroxypropylmethylcellulose phthalate, methacrylic acid-methacrylic acid ester copolymer, polyvinyl acetate-phthalate and cellulose acetate phthalate (Hasegawa, 1985). Various enteric coating materials may be selected on the basis of testing to achieve an enteric coated dosage form designed ab initio to have a preferable combination of dissolution time, coating thicknesses and diametral crushing strength (Porter et al., 1970). On occasion, the performance of an enteric coating may hinge on its permeability (Porter et al., 1981). With such oral drug delivery systems, the drug release process may be initiated by diffuision of aqueous fluids across the enteric coating.

Oral bioavailability of the TAT-tagged eNOS protein of the present invention may be enhanced or mediated using cochleates. Cochleate delivery vehicles are a broad-based enabling technology for the delivery of a wide range of therapeutic products. They are insoluble, non-aqueous, multilayered constructs that are able to survive the stomach, protecting the associated molecules from degradation. Cochleate delivery vehicles are stable phospholipid-cation precipitates composed of simple, naturally occurring materials, for example, phosphatidylserine and calcium (although other multivalent cations can be used). They consist of alternating layers of phospholipid and multivalent cations existing as stacked sheets, or continuous, solid, lipid bilayer sheets rolled up in a spiral configuration, with little or no internal aqueous space. This unique structure provides protection from degradation for associated, or “encochleated,” molecules. Since the entire cochleate structure is a series of solid layers, components within the interior of the cochleate structure remain intact, even though the outer layers of the cochleate may be exposed to harsh environmental conditions or enzymes. This includes protection from digestion in the stomach.

Additional formulations which are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

The actual dosage amount of eNOS of the present invention to be administered to a subject can be determined by physical and physiological factors such as body weight, severity of condition, the type of disease being treated, previous or concurrent therapeutic interventions, idiopathy of the patient and on the route of administration. The number of doses and the period of time over which the dose may be given may vary. The practitioner responsible for administration will, in any event, determine the concentration of active ingredient(s) in a composition and appropriate dose(s), as well as the length of time for administration for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 Imicrogram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

### J. Combination Therapy with eNOS

In order to enhance the effectiveness of treating endothelial dysfunction with the eNOS composition of the present invention, it may be desirable to combine these compositions with other therapies for endothelial dysfunction. For example, one may employ other therapies for treating vascular diseases or conditions therapies such as a conventional therapy or agent, including but not limited to, a pharmacological therapeutic agent, a surgical therapeutic agent (e.g., a surgical procedure) or a combination thereof

It is further contemplated that the eNOS composition of the present invention may be used in conjunction with other therapies such as a chemotherapy in order to increase the efficacy of treatment of angiogenesis such as related to cancer.

This process may involve contacting the cell(s) with the eNOS therapeutic agent of the present invention and a vascular or chemotherapeutic agent at the same time or within a period of time wherein separate administration of the eNOS composition and an agent to a cell, tissue or organism produces a desired therapeutic benefit. The terms “contacted” and “exposed,” when applied to a cell, tissue or organism, are used herein to describe the process by which eNOS and/or an additional therapeutic agent are delivered to a target cell, tissue or organism or are placed in direct juxtaposition with the target cell, tissue or organism. The cell, tissue or organism may be contacted (e.g., by administration) with a single composition or pharmacological formulation that includes both eNOS and one or more agents, or by contacting the cell with two or more distinct compositions or formulations, wherein one composition includes eNOS and the other includes one or more agents such as a vascular or chemotherapeutic agent.

The eNOS composition may precede, be concurrent with and/or follow the other agent(s) by intervals ranging from minutes to weeks. In embodiments where the eNOS composition and other agent(s) are applied separately to a cell, tissue or organism, one would generally ensure that a significant period of time did not expire between the time of each delivery, such that the eNOS composition and agent(s) would still be able to exert an advantageously combined effect on the cell, tissue or organism. For example, in such instances, it is contemplated that one may contact the cell, tissue or organism with two, three, four or more modalities substantially simultaneously (i.e., within less than about a minute). In other aspects, one or more agents may be administered within or from substantially simultaneously, about 1 minute, about 5 minutes, about 10 minutes, about 20 minutes, about 30 minutes, about 45 minutes, about 60 minutes, about 2 hours, or more hours, or about 1 day or more days, or about 4 weeks or more weeks, or about 3 months or more months, or about one or more years, and any range derivable therein, prior to and/or after administering the eNOS therapeutic agent

Typical treatments of subjects having a vascular disease or condition, termed “vascular therapy” include performing surgery on the subject, providing a cardiovascular mechanical prostheses, angioplasty, mechanical circulatory support, coronary artery reperfusion, catheter ablation, or an implantable cardioverter defibrillator to the subject, or administering to the subject a cardiovascular therapeutic agent, thrombolytic agent, or lipid lowering therapy. The term “cardiovascular therapeutic agent” is used in the present disclosure to refer to an agent that is a diuretic, calcium channel blocker, beta blocker, vasodilator, positive inotropic agent, beta-adrenergic agonist, vasopressor, alpha blocker, ACE inhibitor, ANG receptor blocker, ganglion blocking agent, other sympatholytics, andrenergic antagonist, alpha-beta blocker, calcium antagonist, oral anticoagulant, aspirin, warfarin, antiarrhythmic agent (Class I, IB, IC, II, III, or IV), nitroglycerin, magnesium, antiobiotic, antiplatelet agent, statins (HMG-CoA reductases), niacins and other vitamins (including vitamins E and C), or NF-B inhibitors such as leflunomide and its metabolites, resveratrol, oleandrin, and vesnarinone. A mechanical circulatory support may be an intra-aortic balloon counterpulsation or left ventricular assist device. It is further contemplated that the thrombolytic agent comprises heparin, streptokinase, urokinase, tissue plasminogen activator, or a combination thereof.

Thus, it is contemplated that any of these vascular therapies may be administered to a subject in combination with eNOS therapy for the treatment or prevention of a vascular disease or condition. Combination therapy as in the present invention may comprise eNOS as “A” and an additional agent, such as a vascular or chemotherapeutic agent or any other agent involved in endothelial dysfunction as “B”:
A/B/A B/A/B B/B/A A/A/B B/A/A A/B/B B/B/B/A B/B/A/B A/A/B/B A/B/A/B A/B/B/A B/B/A/A B/A/B/A B/A/A/B B/B/B/A A/A/A/B B/A/A/A A/B/A/A A/A/B/A A/B/B/B B/A/B/B B/B/A/B

Administration of the eNOS composition of the present invention to a patient will follow general protocols for the administration of that particular secondary therapy, taking into account the toxicity, if any, of the eNOS therapy. It is expected that the treatment cycles would be repeated as necessary. The compositions employed in the present invention may be administered once or more than once to a subject. Non-limiting examples of therapeutic agents that may be used in conjunction with the eNOS composition of the present invention include an antihyperlipoproteinemic agent, an antiarteriosclerotic agent, an antithrombotic/fibrinolytic agent, a blood coagulant, an antiarrhythmic agent, an antihypertensive agent, a vasopressor, a treatment agent for congestive heart failure, an antianginal agent, an antibacterial agent or a combination thereof, or any other combination as described herein. In some instances, it may be desirable to lower the production of NO thus, inhibitors of eNOS may be applicable in the present invention.

### 1. Antihyperlipoproteinemics

In certain embodiments, administration of an agent that lowers the concentration of one of more blood lipids and/or lipoproteins, known herein as an “antihyperlipoproteinemic,” may be combined with a eNOS therapy according to the present invention, particularly in treatment of athersclerosis and thickenings or blockages of vascular tissues. In certain aspects, an antihyperlipoproteinemic agent may comprise an aryloxyalkanoic/fibric acid derivative, a resin/bile acid sequesterant, a HMG CoA reductase inhibitor, a nicotinic acid derivative, a thyroid hormone or thyroid hormone analog, a miscellaneous agent or a combination thereof.

### a. Aryloxyalkanoic Acid/Fibric Acid Derivatives

Non-limiting examples of aryloxyalkanoic/fibric acid derivatives include beclobrate, enzafibrate, binifibrate, ciprofibrate, clinofibrate, clofibrate (atromide-S), clofibric acid, etofibrate, fenofibrate, gemfibrozil (lobid), nicofibrate, pirifibrate, ronifibrate, simfibrate and theofibrate.

### b. Resins/Bile Acid Sequesterants

Non-limiting examples of resins/bile acid sequesterants include cholestyramine (cholybar, questran), colestipol (colestid) and polidexide.

### c. HMG CoA Reductase Inhibitors

Non-limiting examples of HMG CoA reductase inhibitors include lovastatin (mevacor), pravastatin (pravochol) or simvastatin (zocor).

### d. Nicotinic Acid Derivatives

Non-limiting examples of nicotinic acid derivatives include nicotinate, acepimox, niceritrol, nicoclonate, nicomol and oxiniacic acid.

### e. Thyroid Hormones and Analogs

Non-limiting examples of thyroid hormones and analogs thereof include etoroxate, thyropropic acid and thyroxine.

### f. Miscellaneous Antihyperlipoproteinemics

Non-limiting examples of miscellaneous antihyperlipoproteinemics include acifran, azacosterol, benfluorex, -benzalbutyramide, camitine, chondroitin sulfate, clomestrone, detaxtran, dextran sulfate sodium, 5,8, 11, 14, 17-eicosapentaenoic acid, eritadenine, furazabol, meglutol, melinamide, mytatrienediol, omithine, -oryzanol, pantethine, pentaerythritol tetraacetate, -phenylbutyramide, pirozadil, probucol (lorelco), -sitosterol, sultosilic acid-piperazine salt, tiadenol, triparanol and xenbucin.

### 2. Antiarteriosclerotics

Non-limiting examples of an antiarteriosclerotic include pyridinol carbamate.

### 3. Antithrombotic/Fibrinolytic Agents

In certain embodiments, administration of an agent that aids in the removal or prevention of blood clots may be combined with administration of eNOS therapy, particularly in treatment of atherosclerosis and vasculature (e.g., arterial) blockages. Non-limiting examples of antithrombotic and/or fibrinolytic agents include anticoagulants, anticoagulant antagonists, antiplatelet agents, thrombolytic agents, thrombolytic agent antagonists or combinations thereof.

In certain aspects, antithrombotic agents that can be administered orally, such as, for example, aspirin and wafarin (coumadin), are preferred.

### a. Anticoagulants

A non-limiting example of an anticoagulant include acenocoumarol, ancrod, anisindione, bromindione, clorindione, coumetarol, cyclocumarol, dextran sulfate sodium, dicumarol, diphenadione, ethyl biscoumacetate, ethylidene dicoumarol, fluindione, heparin, hirudin, lyapolate sodium, oxazidione, pentosan polysulfate, phenindione, phenprocoumon, phosvitin, picotamide, tioclomarol and warfarin.

### b. Antiplatelet Agents

Non-limiting examples of antiplatelet agents include aspirin, a dextran, dipyridamole (persantin), heparin, sulfinpyranone (anturane) and ticlopidine (ticlid).

### c. Thrombolytic Agents

Non-limiting examples of thrombolytic agents include tissue plaminogen activator (activase), plasmin, pro-urokinase, urokinase (abbokinase) streptokinase (streptase), anistreplase/APSAC (eminase).

### 4. Antiarrhythmic Agents

Non-limiting examples of antiarrhythmic agents include Class I antiarrythmic agents (sodium channel blockers), Class II antiarrythmic agents (beta-adrenergic blockers), Class II antiarrythmic agents (repolarization prolonging drugs), Class IV antiarrhythmic agents (calcium channel blockers) and miscellaneous antiarrythmic agents.

### a. Sodium Channel Blockers

Non-limiting examples of sodium channel blockers include Class IA, Class IB and Class IC antiarrhythmic agents. Non-limiting examples of Class IA antiarrhythmic agents include disppyramide (norpace), procainamide (pronestyl) and quinidine (quinidex). Non-limiting examples of Class IB antiarrhythmic agents include lidocaine (xylocaine), tocainide (tonocard) and mexiletine (mexitil). Non-limiting examples of Class IC antiarrhythmic agents include encainide (enkaid) and flecainide (tambocor).

### b. Beta Blockers

Non-limiting examples of a beta blocker, otherwise known as a -adrenergic blocker, a -adrenergic antagonist or a Class II antiarrhythmic agent, include acebutolol (sectral), alprenolol, amosulalol, arotinolol, atenolol, befuinolol, betaxolol, bevantolol, bisoprolol, bopindolol, bucumolol, bufetolol, bufuralol, bunitrolol, bupranolol, butidrine hydrochloride, butofilolol, carazolol, carteolol, carvedilol, celiprolol, cetamolol, cloranolol, dilevalol, epanolol, esmolol (brevibloc), indenolol, labetalol, levobunolol, mepindolol, metipranolol, metoprolol, moprolol, nadolol, nadoxolol, nifenalol, nipradilol, oxprenolol, penbutolol, pindolol, practolol, pronethalol, propanolol (inderal), sotalol (betapace), sulfinalol, talinolol, tertatolol, timolol, toliprolol and xibinolol. In certain aspects, the beta blocker comprises an aryloxypropanolamine derivative. Non-limiting examples of aryloxypropanolamine derivatives include acebutolol, alprenolol, arotinolol, atenolol, betaxolol, bevantolol, bisoprolol, bopindolol, bunitrolol, butofilolol, carazolol, carteolol, carvedilol, celiprolol, cetamolol, epanolol, indenolol, mepindolol, metipranolol, metoprolol, moprolol, nadolol, nipradilol, oxprenolol, penbutolol, pindolol, propanolol, talinolol, tertatolol, timolol and toliprolol.

### c. Repolarization Prolonging Agents

Non-limiting examples of an agent that prolong repolarization, also known as a Class III antiarrhythmic agent, include amiodarone (cordarone) and sotalol (betapace).

### d. Calcium Channel Blockers/Antagonist

Non-limiting examples of a calcium channel blocker, otherwise known as a Class IV antiarrythmic agent, include an arylalkylamine (e.g., bepridile, diltiazem, fendiline, gallopamil, prenylamine, terodiline, verapamil), a dihydropyridine derivative (felodipine, isradipine, nicardipine, nifedipine, nimodipine, nisoldipine, nitrendipine) a piperazinde derivative (e.g., cinnarizine, flunarizine, lidoflazine) or a miscellaneous calcium channel blocker such as bencyclane, etafenone, magnesium, mibefradil or perhexiline. In certain embodiments a calcium channel blocker comprises a long-acting dihydropyridine (nifedipine-type) calcium antagonist.

### e. Miscellaneous Antiarrhythmic Agents

Non-limiting examples of miscellaneous antiarrhymic agents include adenosine (adenocard), digoxin (lanoxin), acecainide, ajmaline, amoproxan, aprindine, bretylium tosylate, bunaftine, butobendine, capobenic acid, cifenline, disopyranide, hydroquinidine, indecainide, ipatropium bromide, lidocaine, lorajmine, lorcainide, meobentine, moricizine, pirmenol, prajmaline, propafenone, pyrinoline, quinidine polygalacturonate, quinidine sulfate and viquidil.

### 5. Antihypertensive Agents

Non-limiting examples of antihypertensive agents include sympatholytic, alpha/beta blockers, alpha blockers, anti-angiotensin II agents, beta blockers, calcium channel blockers, vasodilators and miscellaneous antihypertensives.

### a. Alpha Blockers

Non-limiting examples of an alpha blocker, also known as an -adrenergic blocker or an -adrenergic antagonist, include amosulalol, arotinolol, dapiprazole, doxazosin, ergoloid mesylates, fenspiride, indoramin, labetalol, nicergoline, prazosin, terazosin, tolazoline, trimazosin and yohimbine. In certain embodiments, an alpha blocker may comprise a quinazoline derivative. Non-limiting examples of quinazoline derivatives include alfuzosin, bunazosin, doxazosin, prazosin, terazosin and trimazosin.

### b. Alpha/Beta Blockers

In certain embodiments, an antihypertensive agent is both an alpha and beta adrenergic antagonist. Non-limiting examples of an alpha/beta blocker comprise labetalol (normodyne, trandate).

### c. Anti-Angiotension II Agents

Non-limiting examples of anti-angiotension II agents include include angiotensin converting enzyme inhibitors and angiotension II receptor antagonists. Non-limiting examples of angiotension converting enzyme inhibitors (ACE inhibitors) include alacepril, enalapril (vasotec), captopril, cilazapril, delapril, enalaprilat, fosinopril, lisinopril, moveltopril, perindopril, quinapril and ramipril. Non-limiting examples of an angiotensin II receptor blocker, also known as an angiotension II receptor antagonist, an ANG receptor blocker or an ANG-II type-1 receptor blocker (ARBS), include angiocandesartan, eprosaitan, irbesartan, losartan and valsartan.

### d. Sympatholytics

Non-limiting examples of a sympatholytic include a centrally acting sympatholytic or a peripherially acting sympatholytic. Non-limiting examples of a centrally acting sympatholytic, also known as an central nervous system (CNS) sympatholytic, include clonidine (catapres), guanabenz (wytensin) guanfacine (tenex) and methyldopa (aldomet). Non-limiting examples of a peripherally acting sympatholytic include a ganglion blocking agent, an adrenergic neuron blocking agent, a β-adrenergic blocking agent or a alpha1-adrenergic blocking agent. Non-limiting examples of a ganglion blocking agent include mecamylamine (inversine) and trimethaphan (arfonad). Non-limiting of an adrenergic neuron blocking agent include guanethidine (ismelin) and reserpine (serpasil). Non-limiting examples of a β-adrenergic blocker include acenitolol (sectral), atenolol (tenormin), betaxolol (kerlone), carteolol (cartrol), labetalol (norrnodyne, trandate), metoprolol (lopressor), nadanol (corgard), penbutolol (levatol), pindolol (visken), propranolol (inderal) and timolol (blocadren). Non-limiting examples of alphal-adrenergic blocker include prazosin (minipress), doxazocin (cardura) and terazosin (hytrin).

### e. Vasodilators

In certain embodiments eNOS therapy may be combined with a cardiovascular therapeutic agent such as a vasodilator (e.g., a cerebral vasodilator, a coronary vasodilator or a peripheral vasodilator). In certain preferred embodiments, a vasodilator comprises a coronary vasodilator. Non-limiting examples of a coronary vasodilator include amotriphene, bendazol, benfurodil hemisuccinate, benziodarone, chloracizine, chromonar, clobenfurol, clonitrate, dilazep, dipyridamole, droprenilamine, efloxate, erythrityl tetranitrane, etafenone, fendiline, floredil, ganglefene, herestrol bis(-diethylaminoethyl ether), hexobendine, itramin tosylate, khellin, lidoflanine, mannitol hexanitrane, medibazine, nicorglycerin, pentaerythritol tetranitrate, pentrinitrol, perhexiline, pimefylline, trapidil, tricromyl, trimetazidine, trolnitrate phosphate and visnadine.

In certain aspects, a vasodilator may comprise a chronic therapy vasodilator or a hypertensive emergency vasodilator. Non-limiting examples of a chronic therapy vasodilator include hydralazine (apresoline) and minoxidil (loniten). Non-limiting examples of a hypertensive emergency vasodilator include nitroprusside (nipride), diazoxide (hyperstat IV), hydralazine (apresoline), minoxidil (loniten) and verapamil.

### f. Miscellaneous Antihypertensives

Non-limiting examples of miscellaneous antihypertensives include ajmaline, γ-aminobutyric acid, bufeniode, cicletainine, ciclosidomine, a cryptenamine tannate, fenoldopam, flosequinan, ketanserin, mebutamate, mecamylamine, methyldopa, methyl 4-pyridyl ketone thiosemicarbazone, muzolimine, pargyline, pempidine, pinacidil, piperoxan, primaperone, a protoveratrine, raubasine, rescimetol, rilmenidene, saralasin, sodium nitrorusside, ticrynafen, trimethaphan camsylate, tyrosinase and urapidil.

In certain aspects, an antihypertensive may comprise an arylethanolamine derivative, a benzothiadiazine derivative, a N-carboxyalkyl(peptide/lactam) derivative, a dihydropyridine derivative, a guanidine derivative, a hydrazines/phthalazine, an imidazole derivative, a quanternary ammonium compound, a reserpine derivative or a suflonamide derivative.

Arylethanolamine Derivatives. Non-limiting examples of arylethanolamine derivatives include amosulalol, buluralol, dilevalol, labetalol, pronethalol, sotalol and sulfinalol.

Benzothiadiazine Derivatives. Non-limiting examples of benzothiadiazine derivatives include althizide, bendroflumethiazide, benzthiazide, benzylhydrochlorothiazide, buthiazide, chlorothiazide, chlorthalidone, cyclopenthiazide, cyclothiazide, diazoxide, epithiazide, ethiazide, fenquizone, hydrochlorothizide, hydroflumethizide, methyclothiazide, meticrane, metolazone, paraflutizide, polythizide, tetrachlormethiazide and trichlormethiazide.

N-carboxyalkyl(peptide/lactam) Derivatives. Non-limiting examples of N-carboxyalkyl (peptide/lactam) derivatives include alacepril, captopril, cilazapril, delapril, enalapril, enalaprilat, fosinopril, lisinopril, moveltipril, perindopril, quinapril and ramipril.

Dihydropyridine Derivatives. Non-limiting examples of dihydropyridine derivatives include amlodipine, felodipine, isradipine, nicardipine, nifedipine, nilvadipine, nisoldipine and nitrendipine.

Guanidine Derivatives. Non-limiting examples of guanidine derivatives include bethanidine, debrisoquin, guanabenz, guanacline, guanadrel, guanazodine, guanethidine, guanfacine, guanochlor, guanoxabenz and guanoxan.

Hydrazines/Phthalazines. Non-limiting examples of hydrazines/phthalazines include budralazine, cadralazine, dihydralazine, endralazine, hydracarbazine, hydralazine, pheniprazine, pildralazine and todralazine.

Imidazole Derivatives. Non-limiting examples of imidazole derivatives include clonidine, lofexidine, phentolamine, tiamenidine and tolonidine.

Quanternary Ammonium Compounds. Non-limiting examples of quanternary ammonium compounds include azamethonium bromide, chlorisondamine chloride, hexamethonium, pentacynium bis(methylsulfate), pentamethonium bromide, pentolinium tartrate, phenactropinium chloride and trimethidinium methosulfate.

Reserpine Derivatives. Non-limiting examples of reserpine derivatives include bietaserpine, deserpidine, rescinnamine, reserpine and syrosingopine.

Suflonamide Derivatives. Non-limiting examples of sulfonamide derivatives include ambuside, clopamide, furosemide, indapamide, quinethazone, tripamide and xipamide.

### 6. Vasopressors

Vasopressors generally are used to increase blood pressure during shock, which may occur during a surgical procedure. Non-limiting examples of a vasopressor, also known as an antihypotensive, include amezinium methyl sulfate, angiotensin amide, dimetofrine, dopamine, etifelmin, etilefrin, gepefrine, metaraminol, midodrine, norepinephrine, pholedrine and synephrine.

### 7. Treatment Agents for Congestive Heart Failure

Non-limiting examples of agents for the treatment of congestive heart failure include anti-angiotension II agents, afterload-preload reduction treatment, diuretics and inotropic agents.

### a. Afterload-Preload Reduction

In certain embodiments, a subject that can not tolerate an angiotension antagonist may be treated with a combination therapy. Such therapy may combine administration of hydralazine (apresoline) and isosorbide dinitrate (isordil, sorbitrate).

### b. Diuretics

Non-limiting examples of a diuretic include a thiazide or benzothiadiazine derivative (e.g., althiazide, bendroflumethazide, benzthiazide, benzylhydrochlorothiazide, buthiazide, chlorothiazide, chlorothiazide, chlorthalidone, cyclopenthiazide, epithiazide, ethiazide, ethiazide, fenquizone, hydrochlorothiazide, hydroflumethiazide, methyclothiazide, meticrane, metolazone, paraflutizide, polythizide, tetrachloromethiazide, trichlormethiazide), an organomercurial (e.g., chlormerodrin, meralluride, mercamphamide, mercaptomerin sodium, mercumallylic acid, mercumatilin dodium, mercurous chloride, mersalyl), a pteridine (e.g., furterene, triamterene), purines (e.g., acefylline, 7-morpholinomethyltheophylline, pamobrom, protheobromine, theobromine), steroids including aldosterone antagonists (e.g., canrenone, oleandrin, spironolactone), a sulfonamide derivative (e.g., acetazolamide, ambuside, azosemide, bumetanide, butazolamide, chloraminophenamide, clofenamide, clopamide, clorexolone, diphenylmethane-4,4-disulfonamide, disulfamide, ethoxzolamide, furosemide, indapamide, mefruside, methazolamide, piretanide, quinethazone, torasemide, tripamide, xipamide), a uracil (e.g., aminometradine, amisometradine), a potassium sparing antagonist (e.g., amiloride, triamterene)or a miscellaneous diuretic such as aminozine, arbutin, chlorazanil, ethacrynic acid, etozolin, hydracarbazine, isosorbide, mannitol, metochalcone, muzolimine, perhexiline, ticmafen and urea.

### c. Inotropic Agents

Non-limiting examples of a positive inotropic agent, also known as a cardiotonic, include acefylline, an acetyldigitoxin, 2-amino-4-picoline, amrinone, benfuirodil hemisuccinate, bucladesine, cerberosine, camphotamide, convallatoxin, cymarin, denopamine, deslanoside, digitalin, digitalis, digitoxin, digoxin, dobutamine, dopamine, dopexamine, enoximone, erythrophleine, fenalcomine, gitalin, gitoxin, glycocyamine, heptaminol, hydrastinine, ibopamine, a lanatoside, metamivam, milrinone, nerifolin, oleandrin, ouabain, oxyfedrine, prenalterol, proscillaridine, resibufogenin, scillaren, scillarenin, strphanthin, sulmazole, theobromine and xamoterol.

In particular aspects, an intropic agent is a cardiac glycoside, a beta-adrenergic agonist or a phosphodiesterase inhibitor. Non-limiting examples of a cardiac glycoside includes digoxin (lanoxin) and digitoxin (crystodigin). Non-limiting examples of a -adrenergic agonist include albuterol, bambuterol, bitolterol, carbuterol, clenbuterol, clorprenaline, denopamine, dioxethedrine, dobutamine (dobutrex), dopamine (intropin), dopexamine, ephedrine, etafedrine, ethylnorepinephrine, fenoterol, formoterol, hexoprenaline, ibopamine, isoetharine, isoproterenol, mabuterol, metaproterenol, methoxyphenamine, oxyfedrine, pirbuterol, procaterol, protokylol, reproterol, rimiterol, ritodrine, soterenol, terbutaline, tretoquinol, tulobuterol and xamoterol. Non-limiting examples of a phosphodiesterase inhibitor include amrinone (inocor).

### d. Antianginal Agents

Antianginal agents may comprise organonitrates, calcium channel blockers, beta blockers and combinations thereof. Non-limiting examples of organonitrates, also known as nitrovasodilators, include nitroglycerin (nitro-bid, nitrostat), isosorbide dinitrate (isordil, sorbitrate) and amyl nitrate (aspirol, vaporole).

### 8. Inhibitors

In some embodiments of the present invention, eNOS inhibitors can be administered to attenuate the activity of the TAT-tagged eNOS in instances where lowered NO production is desired. Inhibitors contemplated include arginine analogues, such as L-nitroarginine (L-NA), L-N-arginine methyl ester (L-NAME), NG-Monomethyl-L-Arginine Monoacetate (L-NMMA), N5-(1-Iminoethyl)-L-ornithine.dihydrochloride (L-NIO), L-NG-Nitroarginine or NG-Nitro-L-arginine (L-NNA) and Dimethyl-L-arginine; citrulline derivatives, such as Thiocitrulline and (S)-Methylthiocitrulline; indazoles, such as 7-Nitroindazole; imidazol in-N-oxides, such as Potassium carboxy-PTIO; phenylimidazoles, such as TRIM; 21-aminosteroids, such as Tirilazad; biphenyls, such as Diphenyleneiodinium chloride, piperidine derivatives, such as Paroxetine and derivatives of any of the above which are NOS inhibitors or prodrugs thereof.

### 9. Chemotherapeutic Agents

It is also contemplated eNOS therapy of the present invention may be used in combination with chemotherapeutic agents to enhance the efficacy of such agents. Such chemotherapeutic agents may include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, famesyl-protein transferase inhibitors, transplatinum, 5-fluorouracil, vincristine, vinblastine and methotrexate, Temazolomide (an aqueous form of DTIC), or any analog or derivative thereof.

### 10. Surgical Therapeutic Agents

In certain aspects, the secondary therapeutic agent may comprise a surgery of some type, which includes, for example, preventative, diagnostic or staging, curative apd palliative surgery. Surgery, and in particular a curative surgery, may be used in conjunction with other therapies, such as the present invention and one or more other agents.

Such surgical therapeutic agents for vascular diseases and disorders are well known to those of skill in the art, and may comprise, but are not limited to, performing surgery on an organism, providing a cardiovascular mechanical prostheses, angioplasty, coronary artery reperfusion, catheter ablation, providing an implantable cardioverter defibrillator to the subject, mechanical circulatory support or a combination thereof. Non-limiting examples of a mechanical circulatory support that may be used in the present invention comprise an intra-aortic balloon counterpulsation, left ventricular assist device or combination thereof.

### K. Therapeutically Effective Amounts of eNOS

A therapeutically effective amount of eNOS as treatment varies depending upon the host treated and the particular mode of administration. “Therapeutically effective amounts” are those amounts effective to produce beneficial results, particularly with respect to treatment of a vascular disease or condition, in the recipient animal or patient. Such amounts may be initially determined by reviewing the published literature, by conducting in vitro tests or by conducting metabolic studies in healthy experimental animals. Before use in a clinical setting, it may be beneficial to conduct confirmatory studies in an animal model, preferably a widely accepted animal model of the particular disease to be treated. Preferred animal models for use in certain embodiments are rodent models, which are preferred because they are economical to use and, particularly, because the results gained are widely accepted as predictive of clinical value.

An effective amount of a therapeutic agent of the present invention is determined based on the intended goal, for example reduction, suppression or amelioration of a vascular disease or condition. The term “unit dose” refers to physically discrete units suitable for use in a subject, each unit containing a predetermined-quantity of the therapeutic composition calculated to produce the desired responses, discussed above, in association with its administration, i.e., the appropriate route and treatment regimen. The quantity to be administered, both according to number of treatments and unit dose, depends on the subject to be treated, the state of the subject and the protection desired. Precise amounts of the therapeutic composition also depend on the judgment of the practitioner and are particular to each individual.

As is well known in the art, a specific dose level of active compounds such as eNOS, for any particular patient depends upon a variety of factors including the activity of the specific compound employed, the age, body weight, general health, sex, diet, time of administration, route of administration, rate of excretion, drug combination, and the severity of the particular disease undergoing therapy. The person responsible for administration will determine the appropriate dose for the individual subject. Moreover, for human administration, preparations should meet sterility, pyrogenicity, general safety and purity standards as required by FDA Office of Biologics standards.

In one embodiment of the invention the dose range of a eNOS used will be about 0.5 μg/kg body weight to about 0.5 mg/kg body weight to about 500 mg/kg body weight. The term “body weight” is applicable when an animal is being treated. When isolated cells are being treated, “body weight” as used herein should read to mean “total cell weight”. The term “total weight may be used to apply to both isolated cell and animal treatment. All concentrations and treatment levels are expressed as “body weight” or simply “kg” in this application are also considered to cover the analogous “total cell weight” and “total weight” concentrations. However, those of skill will recognize the utility of a variety of dosage range, for example, 1 mg/kg body weight to 450 mg/kg body weight, 2 mg/kg body weight to 400 mg/kg body weighty, 3 mg/kg body weight to 350 mg/kg body weight, 4 mg/kg body weight to 300 mg/kg body weight, 5 mg/kg body weight to 250 mg/kg body weight, 6 mg/kg body weight to 200 mg/kg body weight, 7 mg/kg body weight to 150 mg/kg body weight, 8 mg/kg body weight to 100 mg/kg body weight, or 9 mg/kg body weight to 50 mg/kg body weight. Further, those of skill will recognize that a variety of different dosage levels will be of use, for example, 1 mg/kg, 2 mg/kg, 3 mg/kg, 4 mg/kg, 5 mg/kg, 7.5 mg/kg, 10 mg/kg, 12.5 mg/kg, 15 mg/kg, 17.5 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 120 mg/kg, 140 mg/kg, 150 mg/kg, 160 mg/kg, 180 mg/kg, 200 mg/kg, 225 mg/kg, 250 mg/kg, 275 mg/kg, 300 mg/kg, 325 mg/kg, 350 mg/kg, 375 mg/kg, 400 mg/kg, 450 mg/kg, 500 mg/kg, 550 mg/kg, 600 mg/kg, 700 mg/kg, 750 mg/kg, 800 mg/kg, 900 mg/kg, 1000 mg/kg, 1250 mg/kg, 1500 mg/kg, 1750 mg/kg, 2000 mg/kg, 2500 mg/kg, and/or 3000 mg/kg. Of course, all of these dosages are exemplary, and any dosage in-between these points is also expected to be of use in the invention. Any of the above dosage ranges or dosage levels may be employed for eNOS.

In some embodiments, the present invention contemplates the use of a second agent or therapy for use in treating a vascular disease or condition to be used in conjunction with eNOS for administration to a subject. As long as the dose of the second agent does not exceed previously quoted toxicity levels, the effective amounts of the second agents may simply be defined as those amounts effective to reduce the vascular disease state when administered to an animal in combination with the eNOS This is easily determined by monitoring the animal or patient and measuring those physical and biochemical parameters of health and disease that are indicative of the success of a given treatment. Such methods are routine in animal testing and clinical practice.

### L. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Generation of HT-NOS

The current invention is a TAT-tagged eNOS molecule used to transduce cells in order to correct endothelial dysfunction. The construction, purification, and initial characterization of the TAT-tagged derivative of porcine endothelial nitric oxide synthase (HT-NOS) are described herein. The cloning and engineering of the full-length eNOS cDNA, the affinity purification of the recombinant protein from a baculovirus expression system, the transduction of cultured cells with the purified material, and the assessment of NO production as an indicator of recombinant NOS activity are described.

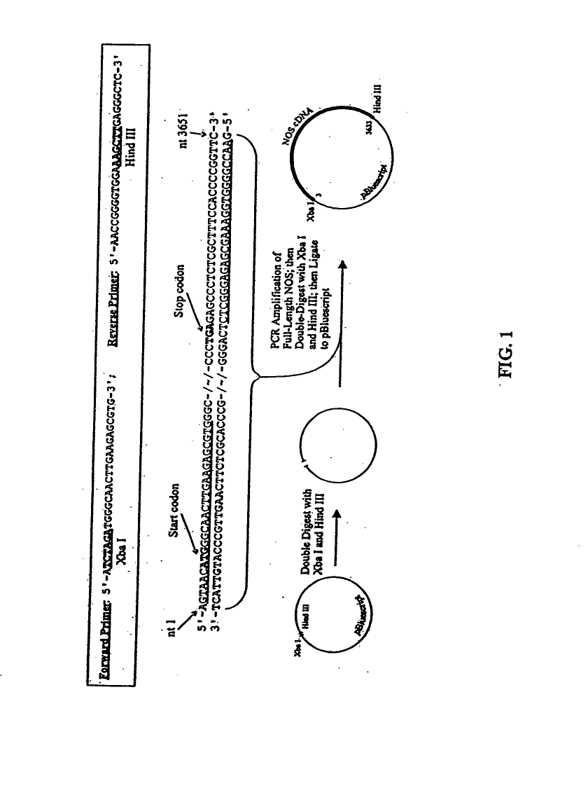

PCR™ Cloning of full-length porcine endothelial NOS. RNA was isolated from aortic endothelial cells from Yucatan miniature swine and cDNA prepared. PCR™ was performed using Pfx polymerase (Stratagene). The forward primer was designed to anneal to the 5′ end of the full-length NOS cDNA, including the initiator methionine start codon (FIG. 1). The forward primer was also designed to introduce a unique Xba I restriction enzyme site immediately prior to the start codon. The reverse primer annealed just past the stop codon of the full-length coding sequence and also introduced a unique Hind III restriction site after the stop codon (FIG. 1). The 3.6 Kb PCR™ product, corresponding to the full-length eNOS coding sequence, was gel-purified, double-digested with Xba I and Hind III, subcloned into the XbaI/Hind III sites of pBluescript, and sequenced in its entirety. This construct was designated “pBS-NOS.”

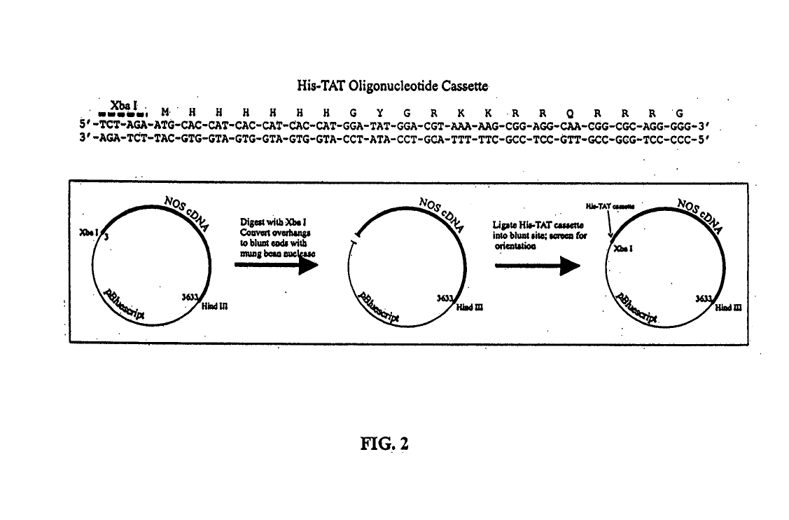

Engineering of His and TAT sequences into eNOS and generation of baculovirus expression vector. Two complimentary oligonucleotides were designed that, when annealed together, made a blunt-ended cassette containing, left to right (5′ to 3′, or amino to carboxy), an Xba I site, then the in-frame codons for an initiator methionine, six histidine residues, a glycine, the 11 amino acid TAT protein transduction domain, and finally, another glycine (FIG. 2). pBS-NOS was digested with Xba I and the sticky-ends were made blunt via the action of mung bean nuclease. The His TAT cassette was ligated into this blunt-ended Xba I site. The entire HT-NOS cDNA was excised from pBS-HT-NOS via double-digest with Xba I and Hind III and subcloned into the Xba/Hind site of the baculovirus transfer vector pBlueBac4.5 (InVitrogen). Recombinant baculovirus was made and immunoblots were used to verify HT-NOS expression.

### Example 2

### Purification of HT-NOS

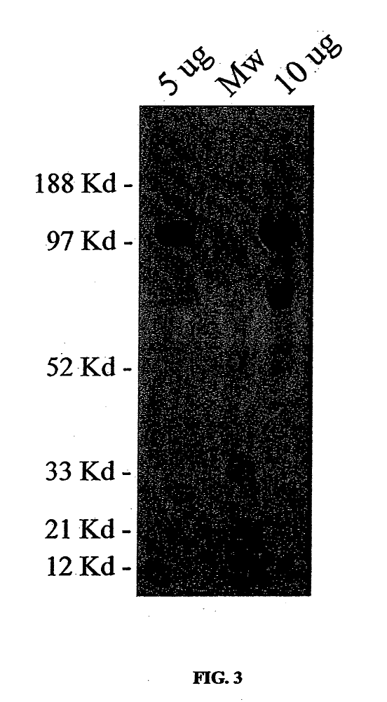

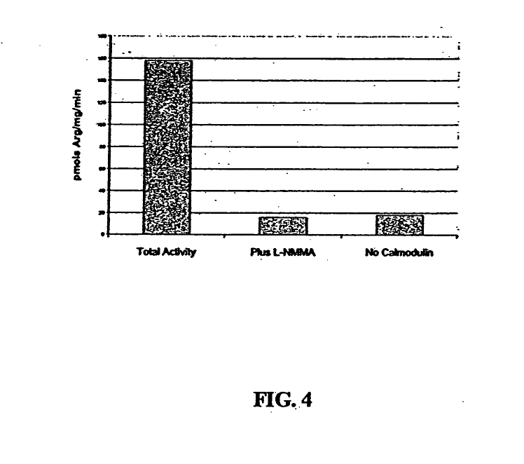

The following protocol was derived in part from Venema et al. (1995) with some modifications and is described in detail herein. Ten confluent T185 cm² flasks of maintenance Hi5 insect cells are split 1:4 into 40 fresh flasks. Cells were infected with 5 μt/flask of the HT-NOS recombinant viral stock (MOI of approximately 1-5) for 1 h with occasional rocking. The media was removed and replaced with 35 ml/flask of fresh media (TNM-FH, 10% FBS, 4 mM glutamine, 40 μg/ml gentamycin, and 3 μg/ml hemin chloride (heme is a NOS prosthetic group and the inclusion of hemin chloride has been shown to be vital for active recombinant NOS; Venema et al., 1995). The cells were incubated at 27° C. for three days, after which time the cells appear heavily infected (pronounced nucleus, enlarged and rounded cells, inhibited cell proliferation were observed). The cells were harvested by striking the flasks on the benchtop followed by centrifugation at 1000×g, for 30 min, at 4° C. All of the following steps were performed at 4° C. The pellets were homogenized in 50 ml (total final volume) of lysis buffer (50 mM Tris, pH 7.0, 0.1% β-mercaptoethanol, 1% Triton X-100), plus protease inhibitors (AEBSF, aprotinin, bestatin, E-64, leupeptin, pepstatin-A, and pefabloc) for 30 min with occasional douncing. The homogenate was centrifuiged at 100,000×g for 45 min and the clear yellowish supernatant was filtered through a cheesecloth and applied to a 1.6-1.8 ml (bed volume) column of ADP-Sepharose at a flow rate of about 0.15 dminute. The column was washed with 25 ml of Wash Buffer (50 mM Tris, pH 7.0, 0.1% β-mercaptoethanol, 20% glycerol, plus protease inhibitors), then with 25 ml of Wash Buffer containing 0.7 M NaCl, 2 mM EGTA plus protease inhibitors, followed by another 25 ml of Wash Buffer (no protease inhibitors). The retained HT-NOS (which appears as a brownish band at the top of the column, presumably due to the heme incorporation into the active recombinant enzyme) was eluted with 5 ml of 10 mM NADPH in Wash Buffer. 0.8 ml fractions were collected and tubes 2-6 were pooled, concentrated to 1 ml final volume with a Centricon-30, aliquoted into 50 μl samples, and frozen at −80° C. Protein was quantitated using BioRad's Bradford reagent. FIG. 3 shows SDS-PAGE analysis of the purified material, using 5 and 10 μg of the purified material. These preparations typically yielded about 700 μg of HT-NOS (from 40 T185 tissue culture flasks). The NT-NOS was at a concentration of 4-6 μM. The purified material was judged to be 85-90% pure based on densitometric (NIH Image) analysis of the digital image (FIG. 4). This degree of purity is in direct accord with published findings of Venema et al. (1995).

The specific activity of the material was determined using a radioactive arginine to citrulline conversion assay. Activity varied, from preparation to preparation, between 150-180 pmols/mg/min, which correlated with published activities for purified recombinant eNOS (Forstermann et al., 1994; Leber et al., 1999). The material exhibited robust activity (FIG. 4) and sensitivity to L-NMMA, a NOS inhibitor, and dependent on the presence of calmodulin in the assay cocktail. Overall, routine purification of adequate amounts of HT-NOS with a high specific activity was accomplished. The engineered His and TAT tags had no apparent effect on the activity of the recombinant enzyme.

### Example 3

### Treatment of Cells with HT-NOS

Initial experiments entailed using NIH 3T3 fibroblasts as the model cells for HT-NOS transduction. These are ideal for preliminary studies because they have no endogenous NOS and they exhibit contact inhibition such that confluent monolayers can be studied over a period of several days. Cells were seeded into a 24 well tissue culture plate and allowed to reach confluence over a period of 2-3 days. At this point, the cells appeared flat and tightly packed together with few cells growing up and over each other. The media was removed and the cells washed twice with serum-free DMEM containing 40 mM HEPES pH 7.4, penicillin and streptomycin. The last wash was removed and replaced with 250 μl of serum-free DMEM containing increasing amounts of purified HT-NOS. The cells were incubated at ambient temperature for 1 hour with occasional rocking, after which they were washed twice with complete media (DMEM plus 10% calf serum and antibiotics) followed by the addition of 2 ml fresh complete media. The cells were maintained in a 37° C., 5% CO₂ incubator for 48 hours, after which the media was removed and the cells washed twice with PBS and dissolved in 100 μl Laemmli SDS-PAGE sample buffer (62.5 mM TrisCl, pH 6.8, 2% SDS, 6 M urea, 160 mM dithiothreitol, 0.001% bromophenol blue). After heating to 70° C. for 30 minutes, 10 μl of each sample were analyzed via SDS-PAGE and immunoblotting. The resulting luminogram from this experiment and the data reveal that HT-NOS can transduce 3T3 cells in a concentration-dependent manner.

To assess that the transduced HT-NOS is biologically active once transduced into cells, the total nitrate/nitrite produced by the cells was quantitated during the 48 hour incubation period post-transduction. This method is superior to a direct measurement of NOS activity (via the [³H]-arginine to [³H]-citrulline conversion assay) for two reasons. First, the direct NOS activity assay detects not only intracellular transduced NOS, but also any extracellular HT-NOS that had simply adsorbed to the cell surface and not been actually transduced. This is because in the in vitro assay, all of the co-factors and substrates that are required by the enzyme for activity are supplied in the reaction mixture; making it difficult to differentiate intracellular HT-NOS from extracellular NOS in these homogenate assays. This problem is resolved when NOS activity by nitrate/nitrite production is assessed. Here, the NO that is produced by the transduced HT-NOS diffuses into the media and is converted to nitrates and nitrites, which are relatively stable (unlike NO) and can accumulate over time (Jain et al., 2001). For the transduced NOS to be active and produce NO in this manner, it must have actually entered the cell since all of the co-factors and substrates (NADPH and arginine) are found endogenously in the intracellular milieu of the transduced cell. The second reason to choose the nitrate/nitrite assay is one of sensitivity. These by-products of NO production are stable and can accumulate over time, thereby amplifying the ability to detect small amounts of active NOS. It was predicted that the transduced cells contained only 1 to 50 ng of NOS per well. Also, such small amounts of material would be difficult to prepare extracts from, and detect NOS activity in a reliable manner using the citrulline assay.

### Example 4

### Nitrate/Nitrate Quanitation

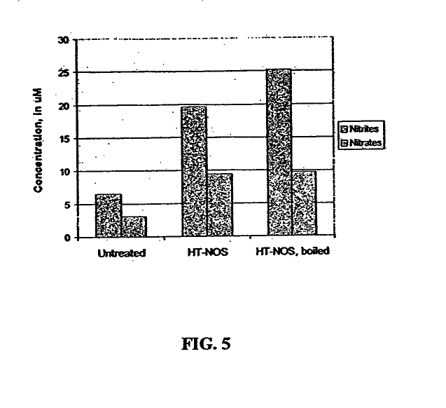

FIG. 5 shows the results of nitrate/nitrite quantitation in media “conditioned” by cells transduced for 48 hours. Cells were transduced as in Example 3, with a few differences. A 6-well plate was used and the cells were incubated with 500 μl of serum-free DMEM containing 0.2 μM HT-NOS. One well was treated with non-denatured HT-NOS; one well was treated with HT-NOS that had been denatured (boiled for 30 seconds prior to dilution into media), and one well was treated with media alone. Following one hour at ambient temperature with occasional rocking, the media was replaced and the cells washed twice with phenol red-free EMEM (which is low in nitrates) containing 10% calf serum and pen/strep. Five milliliters of phenol red-free EMEM plus calf serum and 3 μg/ml hemin chloride was added and the cells incubated at 37° C., 5% CO₂ for 48 hours. Nitrates and nitrites were determined according to the method of Green et al. (1982). Standard curves using sodium nitrite and sodium nitrate were prepared and used to determine nitrate/nitrite levels in the conditioned media. As indicated, control untreated cells had much lower concentrations of nitrates or nitrites compared to treated cells, which had 3-5 times more of the NO metabolites. Furthermore, the samples treated with the denatured preparation had higher levels of nitrates/nitrites, indicating higher NO production.

### Example 5

### HT-NOS Exhibits a Functional Response In Vivo

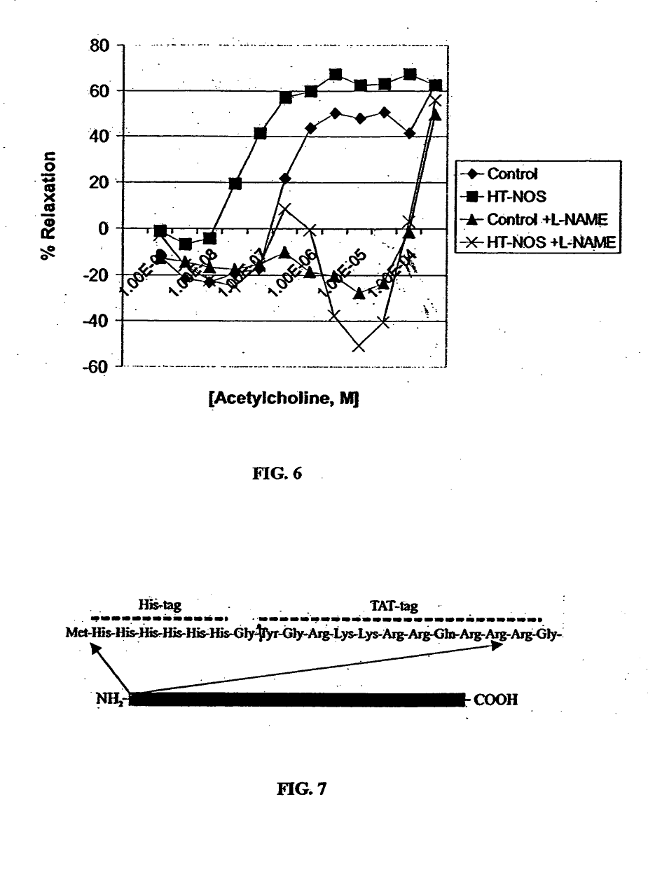

Experiments were designed to demonstrate that HT-NOS exhibits a fuinctional response when administered in vivo. Rats were injected intraperitoneally with 100 μg of purified HT-NOS in phosphate-buffered saline, 10% glycerol. A control rat was injected with PBS/glycerol only. The animals were sacrificed 24 hours later and the abdominal aorta removed and dissected into rings. The rings from the HT-NOS treated rats exhibited enhanced NOS-mediated function compared to the control (FIG. 6), presumably due to HT-NOS transduction into the endothelium. This enhancement was blocked by the NOS inhibitor L-NAME. As shown in FIG. 6, the acetylcholine dose-response curve on phenylephrin-preconstricted rings (vs. % relaxation) is shifted to the left compared to control untreated animals, which would be predicted if the endothelium has increased levels of NOS (the delivered HT-NOS).

### Example 6

### Engineered eNOS

The present invention provides an engineered TAT-tagged version of eNOS. FIG. 7 shows a linear representation of the TAT-hexahistidine tagged eNOS molecule. The recombinant material can be efficiently purified from a baculovirus expression system and exhibits properties expected of eNOS, such as specific activity, inhibition by L-NMMA, dependence on calmodulin, membrane association, and molecular weight. The HT-NOS transduces cells in a manner that is rapid and yields functional NOS activity in cell culture. Studies suggest that HT-NOS can modulate endothelial function when injected into a whole animal.

### Example 7

### HT-NOS Transduction of Human Endothelial Cells

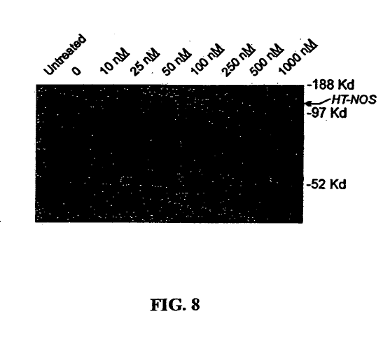

Using cultured endothelial cells, transduction of HT-NOS was assessed by employing methodology as described in the above Examples. Human endothelial cells (HUVEC) were seeded into a 24 well tissue culture plate and allowed to reach confluence over a period of 2-3 days. The media was removed and the cells washed twice with serum-free DMEM containing 40 mM HEPES pH 7.4, penicillin and streptomycin. The last wash was removed and replaced with 250 μl of serum-free DMEM containing increasing amounts of purified HT-NOS. The cells treated were treated with 10 nM, 25 nM, 100 nM, 250 nM 500 nM and 1000 nM of HT-NOS. The cells were incubated at ambient temperature for 1 hour with occasional rocking, after which they were washed twice with complete media (DMEM plus 10% calf serum and antibiotics) followed by the addition of 2 ml fresh complete media. The cells were maintained in a 37° C., 5% CO₂ incubator for 20-24 hours, after which the media was removed and the cells washed twice with PBS and dissolved in 100 μl Laemmli SDS-PAGE sample buffer (62.5 mM TrisCl, pH 6.8, 2% SDS, 6 M urea, 160 mM dithiothreitol, 0.001% bromophenol blue). After heating to 70° C. for 30 minutes, 10 μl of each sample were analyzed via SDS-PAGE and immunoblotting the immunoblot was probed an antipolyhistidine antibody. Coomassie-stained blot was used to verify loading consistency and the immunoblot luminogram revealed that HT-NOS can transduce cultured endothelial cells in a concentration-dependent manner (FIG. 8).

Further experiments are conducted to assess that the transduced HT-NOS is biologically active once transduced into HUVEC cells, as in Example 3, and the total nitrate/nitrite produced by these cells is quantitated during a 48 hour incubation period post-transduction. Nitrate/nitrite quantitation in media “conditioned” by HUvEC cells transduced for 48 hours are conducted as described in Example 4.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### References

The following references, to the extent that they provide exemplary procedural or other details supplementary to those set forth herein, are specifically incorporated herein by reference.
U.S. Pat. No. 4,554,101
U.S. Pat. No. 4,684,611
U.S. Pat. No. 4,879,236
U.S. Pat. No. 4,952,500
U.S. Pat. No. 5,302,523
U.S. Pat. No. 5,322,783
U.S. Pat. No. 5,384,253
U.S. Pat. No. 5,464,765
U.S. Pat. No. 5,538,877
U.S. Pat. No. 5,538,880
U.S. Pat. No. 5,550,318
U.S. Pat. No. 5,563,055
U.S. Pat. No. 5,563,055
U.S. Pat. No. 5,580,859
U.S. Pat. No. 5,589,466
U.S. Pat. No. 5,591,616
U.S. Pat. No. 5,610,042
U.S. Pat. No. 5,656,610
U.S. Pat. No. 5,702,932
U.S. Pat. No. 5,736,524
U.S. Pat. No. 5,780,448
U.S. Pat. No. 5,789,215
U.S. Pat. No. 5,871,986
U.S. Pat. No. 5,945,100
U.S. Pat. No. 5,981,274
U.S. Pat. No. 5,994,624
Alexander et al., Cardiovascular Res., 47:609-617, 2000.
Bachmair et al., Cell, 56:1019-1032, 1989.
Bivalacqua et al., Intl. J. Impotence Res., 12SS- 17, 2000.
Busse and Flemin, Current Hypertension Reports, 1:88-95, 1999.
Cable et al., Cardiovascular Res., 35:553-559, 1997a.
Cable et al., Circulation, 96:73-178; H2575-H2579, 1997b.
Capaldi et al., Biochem. Biophys. Res. Comm., 74(2):425-433, 1977.
Chen and Okayama, Mol. Cell Biol., 7(8):2745-2752, 1987.
Claudius et al., Circulation, 105:1104-1109, 2002.
Darbinian et al., J. Cell. Physiol., 189:334-340, 2001.
De Vriese et al., British J. Pharmacol., 130:963-974, 2000.
Escandon-Calles et al., Endocrine Reviews, 22:36-52, 2001.
Farre and Casado, Hypertension, 38:1400-1405, 2001.
Fechheimer et al., Proc. Natl. Acad. Sci. USA, 84:8463-8467, 1987.
Ferrario et al., Amer. J. Med. Sciences, 323:17-24, 2002.
Forstermann et al., Methods Enzymology, 233:258-265, 1994.
Fraley et al., Proc. Natl. Acad. Sci. USA, 76:3348-3352, 1979.
Frankel and Pabo, Cell, 55:1189-1193, 1988.
Frankel et al, Science, 240:70-73, 1988a.
Frankel et al., Proc. Natl. Acad. Sci. USA, 85:6297-6300, 1988b.
Garcia et al., EMBO J., 7:3143, 1988.
Gopal, Mol. Cell Biol., 5:1188-1190, 1985.
Graham and Van Der Eb, Virology, 52:456-467, 1973.
Green et al., Anal. Biochem., 126:31-138, 1982.
Guerci et al., Diabetes Metab., 27:425-434, 2001 a.
Guerci et al., Diabetes Metab., 27:436-447, 2001b.
Han et al, Molecules and Cells, 10:728-732, 2000.
Harland and Weintraub, J. Cell Biol., 101: 1094-1099, 1985.
Hasegawa, Chem. Pharm. Bull., 33: 1615-1619, 1985.
Hauber et al., J. Virol., 63:1181-1187, 1989.
Hingorani, Atherosclerosis, 154:52 1-527, 2000.
Jain et al., Renal Failure, 23:651-657, 2001.
Jin et al., Free Rad. Biol. Med., 31:1509-1519, 2001.
Johnson et al., In: Biotechnology And Pharmacy, Pezzuto et al (Eds.), Chapman and Hall, NY, 1993.
Kaeppler et al., Plant Cell Reports, 9:415-418, 1990.
Kaneda et al., Science, 243:375-378, 1989.
Kato et al., J. Biol. Chem., 266:3361-3364, 1991.
Kwon et al., FEBS Letters, 485:163-167, 2000.
Kyte and Doolittle, J. Mol Biol., 157(1):105-32, 1982.
Leber et al., J. Biol. Chem., 274:37658-37664, 1999.
Lin et al., Hypertension, 30:307-313, 1997.
Luscher, IJCP Supplement, 117:3-6, 2001.
Maeso et al., General Pharmacol., 34:263-272, 2000.
Marietta, J. Biol. Chem., 268:12231-12234, 1993.
Moncada et al., New Eng. J. Med., 329, pp. 2002-2012 (1993)
Nagahara et al., Nature Medicine, 4:1449-1452, 1998.
Nicolau and Sene, Biochim. Biophys. Acta, 721:185-190, 1982.
Nicolau et al., Methods Enzymol., 149:157-176, 1987.
O'Brien et al., Cardiovascular Res., 42:720-727, 1999.
Olsen et al., Amer. J. Hypertension, 14:861-867, 2001.
Omirulleh et al., Plant Mol. Biol., 21(3):415-28, 1993.
PCT Appl. WO 94/09699
PCT Appl. WO 95/06128
Porter et al., J. Pharm. Pharmacol., 22:42, 1970.
Porter et al., J. Pharm. Pharmacol., 34:5-8, 1981.
Potrykus et al., Mol. Gen. Genet., 199(2):169-77, 1985.
Qian et al., Circulation, 99:2979-2982, 2000.
Remington's Pharmaceutical Sciences, 18th Ed. Mack Printing Company, 1990,
Rippe et al., Mol. Cell Biol., 10:689-695, 1990.
Ruben et al., J. Virol., 63:1-8, 1989.
Sadaie et al., DNA, 8(9):669-74, 1989.
Schwarze et al., Science, 285:1569-1572, 1999.
Schwarze et al., Trends in Pharmacol. Sciences, 21:45-48, 2000.
Suwaidi et al., Circulation, 101:948-954, 2000.
Torchilin et al., PNAS, 98:8786-8791, 2001.
Valiance, et al., J. Royl. Coll. Physician London, 28:209-219, 1994.
Venema et al., Amer. Soc. Biochem. Molec. Biol., 270:14705-14711, 1995.
Wada et al., Nucleic Acids Res., 18:2367-2411, 1990.
Wong et al., Gene, 10:87-94, 1980.
Woodman et al., Am J. Physiol., 273(6 Pt 2):H2575-9, 1997.
Yang et al., Molec. Pharmacol., 61:269-276, 2002.

1. A method for treating a vascular disease or condition comprising providing to a cell an endothelial nitric oxide synthase (eNOS) comprising a TAT protein transduction domain, wherein the eNOS increases nitric oxide production in a cell.

2. The method of claim 1, wherein eNOS further comprises a hexa-histidine domain.

3. The method of claim 1, wherein the vascular disease or condition is heart disease, hypertension, diabetes, atherosclerosis, hyperlipidemia, erectile dysfunction or arthritis.

4. The method of claim 1, wherein the cell is a vascular cell.

5. The method of claim 4, wherein the vascular cell is an endothelial cell.

6. The method of claim 4, wherein the vascular cell is vascular smooth muscle cell.

7. The method of claim 1, wherein the cell is located in a cell culture.

8. The method of claim 1, wherein the cell is located in a tissue culture.

9. The method of claim 1, wherein the cell is located in the vasculature of a mammal.

10. The method of claim 1, further comprising detecting eNOS comprising a TAT protein transduction domain in a vascular cell by immunoblotting.

11. A method for detecting a vascular disease in a subject comprising: a) obtaining a vascular cell sample from a subject; and b) analyzing the cell sample for nitric oxide production by endothelial nitric oxide synthase, wherein a decrease in nitric oxide production as compared to a vascular cell sample from a healthy subject, indicates a vascular disease.

12. The method of claim 11, wherein the subject is a mammal.

13. The method of claim 12, wherein the mammal is a human.

14. The method of claim 11, wherein the vascular cell sample is vascular smooth muscle cell sample.

15. The method of claim 11, wherein the vascular cell sample is a heart disease cell sample, a hypertension cell sample, a diabetes cell sample, a atherosclerosis cell sample or a hyperlipidemia cell sample.

16. The method of claim 11, wherein nitric oxide production is analyzed by a nitrite assay.

17. The method of claim 11, wherein nitric oxide production is analyzed by a nitrate assay.

18. The method of claim 11, wherein nitric oxide production is analyzed by a cGMP assay.

19. The method of claim 11, wherein the nitric oxide production is analyzed by monitoring blood pressure, blood flow, and improvements in vascular reactivity.

20. A method for treating a subject having a vascular disease or condition comprising administering to the subject a therapeutic effective amount of an endothelial nitric oxide synthase comprising a TAT protein transduction domain.

21. The method of claim 20, wherein the subject is a mammal.

22. The method of claim 21, wherein the mammal is a human.

23. The method of claim 20, wherein the vascular disease or condition is heart disease, hypertension, diabetes, atherosclerosis, hyperlipidemia, erectile dysfunction or arthritis.

24. The method of claim 20, wherein administering is intravenously, intraarterially, subcutaneously, orally or topically.

25. A method for assessing the efficacy of eNOS comprising a TAT protein transduction domain as a vascular cell therapy comprising:
a) administering a endothelial nitric oxide synthase protein comprising a TAT protein transduction domain to a subject having a vascular disease or condition; and
b) determining nitric oxide production;
wherein an increase in the nitric oxide production as compared to the nitric oxide production in a vascular cell prior to administering eNOS comprising a TAT protein transduction domain, indicates that the vascular cell therapy is effective.

26. The method of claim 25, wherein nitric oxide production is determined by a nitrite assay.

27. The method of claim 25, wherein nitric oxide production is determined by a nitrate assay.

28. The method of claim 25, wherein nitric oxide production is determined by a cGMP assay.

29. The method of claim 25, wherein the nitric oxide production is determined by monitoring blood pressure, blood flow, and improvements in vascular reactivity.

30. The method of claim 25, wherein the vascular disease or condition is heart disease, hypertension, diabetes, atherosclerosis, hyperlipidemia, erectile dysfunction or arthritis.

31. The method of claim 25, wherein the vascular cell is a vascular smooth muscle cell.

32. The method of claim 25, wherein the vascular cell is an endothelial cell.

33. An endothelial nitric oxide synthase comprising a TAT protein transduction domain.

34. The endothelail nitric oxide synthase of claim 33 further comprising a hexa-histidine domain.

35. A pharmaceutical composition comprising a endothelial nitric oxide synthase containing a TAT protein transduction domain.
